(19) United States
(12) Patent Application Publication
(10) Pub. No.: US 2025/0283144 A1
(43) Pub. Date: Sep. 11, 2025

(54) ELEVATION OF GLYCOSAMINOGLYCANS IN SUBJECTS WITHOUT MUCOPOLYSACCHARIDOSIS

(71) Applicant: THE NEMOURS FOUNDATION, Jacksonville, FL (US)

(72) Inventors: Shunji TOMATSU, Wilmington, DE (US); Seiji YAMAGUCHI, Izumo, Shimane (JP); Hironori KOBAYASHI, Izumo, Shimane (JP)

(73) Assignee: THE NEMOURS FOUNDATION, Jacksonville, FL (US)

(21) Appl. No.: 18/265,076

(22) PCT Filed: Dec. 7, 2021
(86) PCT No.: PCT/US2021/062229

§ 371 Date: Jun. 2, 2023

Related U.S. Application Data
(60) Provisional application No. 63/122,219, filed on Dec. 7, 2020.

(51) Int. Cl. C12Q 1/527; C12Q 1/34

(52) U.S. Cl. CPC C12Q 1/527; C12Q 1/34; G01N 2400/40; G01N 2800/28; G01N 2800/52

(57) ABSTRACT

Provided are methods of using glycosaminoglycans (GAGs) levels in a biological sample as a means to diagnose, assess prognosis, and ascertain whether treatment is efficacious in a wide variety of diseases other than mucopolysaccharidoses (MPS). In certain embodiments, the methods relate to clinical diagnosis of viral or non-viral encephalopathy.

### CROSS-REFERENCE TO RELATED APPLICATIONS

This application claims the benefit of the filing date of U.S. Provisional Patent Application No. 63/122,219, filed Dec. 7, 2020, which is hereby incorporated in its entirety for all purposes.

### REFERENCE TO GOVERNMENT GRANT

The invention was made with government support under grant Nos. P30GM114736 and 1R01HD065767 awarded by the National Institutes of Health. The government has certain rights in the invention.

### FIELD OF DISCLOSURE

The present disclosure relates to screening for a disease or disorder by detecting an elevated level of at least one glycosaminoglycan in a biological sample, and treatment of individuals afflicted with the disease or disorder, as well as to methods of assessing prognosis and monitoring treatment efficacy.

### BACKGROUND OF THE DISCLOSURE

In humans, carbohydrates exist in the form of sulfated polysaccharide chains called glycosaminoglycans (GAGs). These GAG chains attach to core proteins, forming proteoglycans (PGs), which have a variety of functions, including cell signaling, stimulating growth and development, and extracellular matrix (ECM) hydration. The core proteins of proteoglycans can be transmembrane proteins; therefore, GAGs can be a part of the ECM or part of the glycocalyx. GAGs include heparan sulfate (HS), chondroitin sulfate (CS), dermatan sulfate (DS), keratan sulfate (KS), and hyaluronic acid (HA). HA differs from the other GAGs, as it is neither sulfated nor linked to a core protein [1].

Each GAG chain is found in different locations and has roles in the brain and central nervous system (CNS). Chondroitin sulfate proteoglycans (CSPGs), which contain both DS and CS, are the most abundant proteoglycan in the central nervous system (CNS). The PGs of the letican family, which contain mainly CS and KS [2], are the main constituents of the brain ECM. These PGs bind with HA and other link proteins in the brain ECM to bind with neurons [3]. CSPGs typically act as barrier molecules, directing axon growth and synapse formation. Similarly, keratan sulfate proteoglycans (KSPGs) also are mainly involved in neuronal outgrowth and synapse organization in the CNS. KSPGs also play a role in neurotransmission and nerve regeneration [2]. In fact, both KS and CS are known to play a role in glial scarring and regeneration following brain injury. Both CS and DS have been shown to bind to morphogens, making them essential in CNS development and to mediate cell proliferation [4]. The main function of HA in the CNS is its structural role in the formation of the brain ECM, but it has also been shown to bind to growth factors and cytokines. Additionally, low molecular weight HA is involved with inflammation after CNS injury [4]. Finally, heparan sulfate proteoglycans (HSPGs) are a major component of the brain's vascular basement membrane [5]; they bind with signaling molecules, preventing their degradation and creating storage pools, and HSPGs form ternary complexes with signaling molecules and their receptors to promote signaling.

Mucopolysaccharidoses (MPS) are a group of rare inherited metabolic disorders in which patients have a deficiency of the lysosomal enzyme required for degradation of one or more GAG, leading to an accumulation of GAGs in the lysosomes. This accumulation interrupts normal cell physiology, resulting in a complex syndrome with symptoms including skeletal dysplasia, organ dysfunction, developmental delay, cognitive impairment, hearing loss, and joint rigidity or hypermobility. Currently, enzyme replacement therapy and hemopoietic stem cell transplantation are available clinically for MPS. Both treatments provide a better prognosis if patients are treated at an early age. To identify and treat patients as soon as possible, HS, DS, and KS are commonly used as biomarkers for high-risk or newborn screening of MPS [6, 7].

Previous studies have shown that some proteoglycans (PGs) are elevated or altered in various specimens (e.g., urine, blood, cerebrospinal fluid (CSF), tissues, etc.) in some diseases or conditions [8, 9]. For example, the DSPG endocan has been shown to be elevated in patients with stable chronic obstructive pulmonary disease (COPD) [8], and syndecan-4, a HSPG, has been shown to be elevated in response to bacterial pneumonia [9]. Other studies demonstrated the elevation of specific GAGs in some diseases or conditions, mainly in adulthood. For instance, GAGs constitute a large part of the endothelial glycocalyx in the vascular lumen, which has been shown to be perturbed in systematic inflammation illnesses, such as respiratory failure or septic shock, leading to an increase in serum GAGs [10, 11]. The endothelial glycocalyx has also been implicated in post-cardiac arrest syndrome, indicating that cardiac arrest or resuscitations can result in shedding of syndecan-1, HS, and HA into blood circulation. Additionally, surviving patients had lower HS and syndecan-1 levels than deceased patients, indicating that the extent of glycocalyx perturbation and corresponding levels of GAGs in blood could indicate prognosis in these patients [12].

In the human body, GAGs are present in proteoglycans, which play critical physiological roles in a variety of tissues. There is a need in the art to investigate the possible relationship of specific GAGs in a biological sample regarding a wide range of common diseases and inherited metabolic disorders, especially in childhood.

### SUMMARY OF THE DISCLOSURE

The present disclosure relates to glycosaminoglycans as biomarkers for diseases or conditions other than mucopolysaccharidoses (MPS).

The present disclosure provides methods of using glycosaminoglycans (GAGs) levels as a means to diagnose, assess prognosis, and ascertain whether treatment is efficacious in a wide variety of diseases, other than mucopolysaccharidoses (MPS).

As envisioned in the present disclosure regarding the materials and methods described, in one aspect, the embodiments of the disclosure comprise the components and/or steps disclosed herein. In another aspect, the embodiments of the disclosure consist essentially of the components and/or steps disclosed herein. In yet another aspect, the embodiments of the disclosure consist of the components and/or steps disclosed herein. The therapeutic methods and compositions used in these methods as described herein can be alternatively considered as a use of the level of glycosaminoglycans in a biological sample for use in diagnosing, assessing prognosis, and/or monitoring treatment efficacy in a patient in need thereof.

### FIGURES

The patent or application file contains at least one drawing executed in color. Copies of this patent or patent application publication with color drawing(s) will be provided by the Office upon request and payment of the necessary fee.

FIG. 1, comprised of FIGS. 1A-1F, illustrates the distributions of glycosaminoglycans (GAGs) in viral encephalopathy patients, non-viral encephalopathy patients, and control patients in each age group (age in years). FIG. 1A depicts data for ΔDiHS-0S. FIG. 1B depicts data for ΔDiHS-NS. FIG. 1C depicts data for ΔDi-4S. FIG. 1D depicts data for mono-sulfated KS. FIG. 1E depicts data for di-sulfated KS. FIG. 1F reflects the ratio of di-sulfated KS to total KS. Statistically significant differences for a two-tailed t-test are marked in each figure with an asterisk. The y-axis is split in FIGS. 1B and 1C due to extreme outliers in the data. For each age group, the data from left to right are control, non-viral encephalopathy, and viral encephalopathy.

FIG. 2, comprised of FIGS. 2A-2F, illustrates the distributions of glycosaminoglycans (GAGs) in patients diagnosed with bacterial infection (sepsis or meningitis) and control patients. The data from left to right in each figure are control and bacterial infection. FIG. 2A depicts data for ΔDiHS-0S. FIG. 2B depicts data for ΔDiHS-NS. FIG. 2C depicts data for ΔDi-4S. FIG. 2D depicts data for mono-sulfated KS. FIG. 2E depicts data for di-sulfated KS. FIG. 2F reflects the ratio of di-sulfated KS to total KS (%). The single dots represent data outliers. “X”: indicates the average (mean). The horizontal line in each box indicates the median. Statistically significant differences or trends of bacterial infection vs. other for a two-tailed t-test are: 0.009899 (DiHS-0S); 0.311683 (DiHS-NS); 0.511314 (Di4S); 0.07868 (monosulfated KS); 0.282426 (DiS KS); and 0.052496 (DiKS/total KS).

### DEFINITIONS

Unless defined otherwise, all technical and scientific terms used herein have the same meaning as commonly understood by one of ordinary skill in the art to which the disclosure pertains. Although any methods and materials similar or equivalent to those described herein can be used in practice for testing of the present disclosure, the preferred materials and methods are described herein. The following terminology will have the indicated meanings unless specifically indicated otherwise.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element. Thus, recitation of “a cell”, for example, includes a plurality of the cells of the same type.

“About” as used herein when referring to a measurable value such as an amount, a temporal duration, and the like, is meant to encompass variations of +/−20% or +/−10%, or +/−5%, or +/−1%, and +/−0.1% from the specified value, as such variations are appropriate to perform the disclosed methods.

By KS is meant keratan sulfate.

By DS is meant dermatan sulfate.

By ΔDiHS-0S is meant 2-acetamido-2-deoxy-4-O-(4-deoxy-a-L-threo-hex-4-enopyranosyluronic acid)-D-glucose.

By ΔDiHS-NS is meant 2-deoxy-2-sulfamino-4-(4-deoxy-a-L-threo-hex-4-enopyranosyluronic acid)-D-glucose.

By ΔDi-4S is meant 2-acetamido-2-deoxy-4-O-(4-deoxy-α-L-threo-hex-4-enopyranosyluronic acid)-4-O-sulfo-D-glucose.

By mono-S KS or mono-sulfated KS is meant mono-sulfated keratan sulfate (Galβ1-4GlcNAc(6S)).

By di-S KS or di-sulfated KS is meant di-sulfated keratan sulfate (Gal(6S) #1-4GlcNAc(6S)).

An “effective amount” as used herein, means an amount that provides a therapeutic or prophylactic benefit.

“Parenteral” administration of a composition includes, e.g., subcutaneous (s.c.), intravenous (i.v.), intramuscular (i.m.), intraperitoneal, or intrascisernal injection, or infusion techniques.

The terms “patient,” “subject,” “individual,” and the like are used interchangeably herein, and can include a human being.

To “treat” a disease as the term is used herein, means to reduce the frequency or severity of at least one sign or symptom of a disease or disorder experienced by a subject.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

By “control” or “control sample,” is meant a sample from a subject that does not have elevated glycosaminoglycans (GAGs). A subject that does not have elevated GAGs and has no findings or unique conditions, is also referred to herein as a “control subject” or “control patient.” A “control” can also refer to a value or range of values derived from analysis of pooled control samples. A control or control sample is preferably age-matched with the test subject sample. An “elevated glycosaminoglycan” refers to any level of at least one standard deviation or at least two standard deviations above the mean normal level for a specific GAG. In an embodiment, an “elevated glycosaminoglycan” refers to any level of at least two standard deviations above the mean normal level for a specific GAG. A “normal level” is the level of a GAG from a control, and also encompasses the level or range of levels derived from the analysis of pooled control samples.

By “sample” or “test sample” as used herein means a biological material isolated from an individual. The test sample may contain any biological material such as a bodily fluid or body tissue suitable for detecting the desired biomarkers.

A “biological sample” for measuring GAGs is a body fluid sample such as whole blood, plasma, serum, urine, cerebrospinal fluid (CSF), or other bodily fluids, or a body tissue sample. No particular limitation is placed on the nature of the sample so long as it contains mucopolysaccharides. In one embodiment, a blood sample may be in dried form, e.g., blood spot.

By “liquid chromatography-tandem mass spectroscopy analysis” or “LC-MS/MS” is meant a chemical analytical technique that combines liquid chromatography with tandem mass spectrometry for quantification of substances in a mixture of substances. By “tandem mass spectrometry,” also known as “MS/MS,” is meant a multi-step mass spectrometry technique using a separate mass spectroscope for each step or by using the same spectroscope to perform steps sequentially, typically with some form of fragmentation occurring in between the steps.

### DETAILED DESCRIPTION OF THE DISCLOSURE

The present disclosure describes the investigation of serum GAG levels in a subset of patients with conditions other than mucopolysaccharidoses (MPS), who had elevated GAGs because of the deficiency of GAG degrading enzyme. As disclosed herein, serum GAGs were elevated unexpectedly in a variety of diseases and conditions. The largest group of patients had a clinical diagnosis of viral or non-viral encephalopathy. Clinical diagnoses and conditions also included epilepsy, fatty acid metabolism disorders, respiratory and renal disorders, liver disorders, hypoglycemia, developmental disorders, hyperCKemia, myopathy, acidosis, and vomiting disorders. The results disclosed herein demonstrate that GAGs in blood are may be biomarkers in more common disorders, in addition to MPS. As further disclosed herein, patients with conditions other than mucopolysaccharidoses (MPS) also had elevated GAG levels in (CSF.

Accordingly, the disclosure describes methods of using glycosaminoglycans (GAGs) levels as a means to diagnose, assess prognosis, and ascertain whether treatment is efficacious in a wide variety of diseases other than mucopolysaccharidoses (MPS). In certain embodiments, the methods related to clinical diagnosis of viral or non-viral encephalopathy. Additional diseases and conditions included epilepsy, fatty acid metabolism disorders, respiratory disorders, renal disorders, viral infections, liver disorders, hypoglycemia, developmental disorders, paucisymptomatic hyperCKemia (a condition indicating the presence of non-specific symptoms such as myalgias, cramps, and/or fatigue with physical activity), isolated or asymptomatic hyperCKemia (a condition generally characterized by elevated levels of the enzyme creatine kinase), heart disease, myopathy, acidosis, and vomiting disorders.

The method of determining GAG levels in a subject described herein can be used to assess the presence of a condition. The method can also be used to assess the severity of the condition. Additionally, the method of determining the GAG levels in a subject can be used to assess responsiveness to one or more therapies to treat the condition, disorder, or infection resulting in the elevated GAG level(s).

In the method of determining GAG levels in a subject used to assess the presence of a condition, the method is practiced with a subject that does not have a MPS disease. The subject can be determined to be afflicted with a non-MPS disease or disorder when the level of a least one disaccharide obtained from a glycosaminoglycan in a biological sample from the subject containing glycosaminoglycans is elevated compared to the level of the same disaccharide in a biological sample from a control. In some embodiments, the control biological sample is the same type, e.g., blood, blood spot or CSF, as the biological sample from the subject. In some embodiments, the control comprises an age-matched control. The diagnosis of a non-MPS disease or disorder can be further confirmed by performing one or more diagnosis procedures known in the art, such as procedures to diagnose encephalopathy as viral or non-viral. The subject diagnosed with a non-MPS disease or disorder in the method of the disclosure can then be administered a treatment known in the art for the non-MPS disease or disorder.

The method can be used, for example, to assist in diagnosing the presence of and severity of any one of the following conditions, disorders or infections: viral encephalopathy, non-viral encephalopathy, epilepsy, a fatty acid metabolism disorder, a respiratory disorder, a renal disorder, a viral infection, a liver disorder, hypoglycemia, a developmental disorder, a hyperCKemia, a heart disease, myopathy, acidosis, and a vomiting disorder, and combinations of these disorders and conditions as exemplified in Table 1. Additional disorders for diagnosis and treatment monitoring include hypoglycemia, hypoglycemic attack, rhabdomyolysis, hypernatremia, ketotic hypoglycemia, impaired consciousness, hypoglycemia, hypophosphatasia, West syndrome, hyperammonemia, recurrent myopathy, gall stone, choledocholithiasis, and multiple organ failure

Encephalopathy is any brain disease or injury that affects the structure or function of the brain. Many events can cause encephalopathy, including infection, tumor, or stroke. Encephalopathy conditions contemplated for use with these methods include hypoglycemia encephalopathy, acute encephalopathy, influenza-induced encephalopathy, acute encephalopathy disseminated intravascular coagulopathy (DIC), acute encephalopathy with seizures, Leigh encephalopathy, hypoxic-ischemic encephalopathy, Ifosfamide encephalopathy, lissencephaly with Infantile spasms, acute viral encephalopathy, acute focal bacterial nephritis encephalopathy, encephalopathy caused by an unknown virus, hypoxic-ischemic encephalopathy, CPA post influenza A, leukoencephalopathy, medium-chain acyl-CoA dehydrogenase (MCAD) deficiency, Reye's syndrome, and CADASIL syndrome (Cerebral autosomal dominant arteriopathy with subcortical infarcts and leukoencephalopathy). Non-viral encephalopathy diagnoses include megalencephaly, hypoglycemia encephalopathy, epileptic encephalopathy, hypoxic-ischemic encephalopathy, Leigh syndrome, periventricular leukomalacia, ifosfamide-induced encephalopathy, acute focal bacterial nephritis (AFBN) encephalopathy, and leukoencephalopathy.

In certain embodiments, viral encephalopathy is one of respiratory syncytial virus (RSV), influenza A, influenza B, rotavirus, human herpes virus 6 (HHV-6), and norovirus.

In certain embodiments, non-viral encephalopathy is one of megalencephaly, hypoglycemia encephalopathy, epileptic encephalopathy, hypoxic-ischemic encephalopathy, Leigh syndrome, periventricular leukomalacia, ifosfamide-induced encephalopathy, acute focal bacterial nephritis (AFBN) encephalopathy, and leukoencephalopathy.

In certain embodiments, viral encephalopathy is indicated in a subject by an elevation in ΔDiHS-0S relative to a control group. In certain embodiments, the subject is 0 to 2.9, 5 to 9.9, or 10 to 14.9 years old.

In certain embodiments, non-viral encephalopathy is indicated in a subject. by an elevation in ΔDiHS-0S relative to a control group. In certain embodiments, the subject is 0 to 2.9 years old.

In certain embodiments, non-viral encephalopathy is indicated in a subject by an elevation in ΔDiHS-NS relative to a control group. In certain embodiments, the subject is 5 to 9.9 years old, or 15+ years old.

In certain embodiments, viral encephalopathy is indicated in a subject by an elevation in ΔDi-4S relative to a control group. In certain embodiments, the subject is 10 to 14.9 years old.

In certain embodiments, non-viral encephalopathy is indicated in a subject by an elevation in ΔDi-4S relative to a control group. In certain embodiments, the subject is 0 to 2.9 years old or 15+ years old.

In certain embodiments, non-viral encephalopathy is indicated in a subject by an elevation in mono-sulfated KS relative to a control group. In certain embodiments, the subject is 0 to 2.9 years old.

In certain embodiments, viral encephalopathy is indicated in a subject by an elevation in di-sulfated KS relative to a control group. In certain embodiments, the subject is 0 to 2.9 year old or 10 to 14.9 years old.

In certain embodiments, non-viral encephalopathy is indicated in a subject by an elevation in di-sulfated KS relative to a control group. In certain embodiments, the subject is 0 to 2.9 years old or 10 to 14.9 years old.

In certain embodiments, non-viral encephalopathy is indicated in a subject by an elevation in the ratio of di-sulfated KS to total KS compared to a control. In certain embodiments, viral encephalopathy is indicated in a subject by an elevation in the ratio of di-sulfated KS to total KS compared to a control. In these embodiments, the subject can be 0 to 2.9 years old, 5 to 9.9 years old or 10 to 14.9 years old.

In certain embodiments, there was a significant difference in ΔDiHS-0S for the 10-14.9 age group and in mono-sulfated KS for the 0-2.9 age group for viral encephalopathy relative to non-viral encephalopathy. Thus, a differential diagnosis of viral encephalopathy rather than non-viral encephalopathy may be indicated by the elevation in ΔDiHS-0S for the 10 to 14.9 years old group and in mono-sulfated KS for the 0 to 2.9-year-old group.

Fatty acid metabolism disorders contemplated for use with these methods include carnitine palmitoyltransferase 2 (CPT2) deficiency, secondary carnitine deficiency, carnitine deficiency, hypocarnitinemia, VLCAD (very long-chain acyl-CoA dehydrogenase) deficiency, glutaric academia type II (GA II or GA-2), hyperlactatemia, FBPase deficiency (fructose-1,6-bisphosphatase deficiency), and hyperCKemia. In certain embodiments, fatty acid metabolism disorders is one of carnitine deficiency, Reye's syndrome, carnitine palmitoyltransferase 2 (CPT2) deficiency, medium-chain acyl-CoA dehydrogenase (MCAD) deficiency, and very-long-chain acyl-CoA dehydrogenase (VLCAD) deficiency).

Seizure and epilepsy disorders contemplated for use with these methods include generalized seizure disorder, hypoglycemia-related convulsions, convulsive seizures, febrile convulsion, acute encephalopathy with biphasic seizures, and late reduced diffusion afebrile convulsion, epileptic encephalopathy, complex partial status epilepsy, and tonic-clonic seizure. In certain embodiments, epilepsy is one of epilepsy, West syndrome, tonic-clonic seizures, and febrile seizures.

Heart disease or condition contemplated for use with these methods include myocarditis, pulmonary hypertension crisis, congenital heart disease, and multi-organ failure. In certain embodiments, heart conditions include hypertrophic cardiomyopathy, abnormal ECG (electrocardiogram also referred to as EKG), mitral regurgitation (MR), myocarditis, and ventricular tachycardia. In certain embodiments, heart conditions is one of hypertrophic cardiomyopathy, abnormal ECG, mitral regurgitation (MR), myocarditis, and ventricular tachycardia.

A respiratory disorder contemplated for use with these methods includes chronic lung disease, asthma, chronic obstructive airway disease (COPD), rhabdomyolysis, and multi-organ failure.

Renal disorders contemplated for use with these methods include renal failure, kidney stones, fulminant hepatic failure, and multi-organ failure.

In certain embodiments, respiratory or renal disorders is one of pneumonia, asthma, bronchitis, chronic obstructive pulmonary disease (COPD), and rhabdomyolysis.

Virus, bacterial and other infections contemplated for use with these methods include respiratory syncytial virus (RSV), hepatitis, rotavirus, norovirus, influenza (e.g., influenza A and influenza B), human herpes virus 6 (HHV-6), norovirus encephalitis, adenovirus, unknown virus related hepatitis (herpangina), and mycoplasma pneumonia. In certain embodiments, a viral infection is one of rotavirus, hand-foot-mouth disease, and influenza.

Vomiting disorders contemplated for use with these methods include cyclic vomiting, virus-related vomiting, acetonemic vomiting, cyclic vomiting, and a bilious attack-induced vomiting. In certain embodiments, vomiting disorder is cyclic vomiting syndrome.

Liver disorders contemplated for use with these methods include recurrent liver dysfunction, herpangina, hepatitis, jaundice, hyperbilirubinemia, liver dysfunction and multi-organ failure. In certain embodiments, liver disorders include jaundice, hyperbilirubinemia, and liver dysfunction. In certain embodiments, a liver disorder is one of jaundice, hyperbilirubinemia, and liver dysfunction.

In certain embodiments, acidosis is one of glutaric acidemia II (GAII) and methylmalonic acidemia.

Treatments for the above-described disorders and diseases are well known in the art (see, for instance, Merck Manual of Diaagnosis and Therapy, 20^{th} Edition, 2018). For many disorders and diseases, such as encephalopathies, treatments generally depend on the underlying cause of the disorders and disease. Treatments include, but are not limited to, anti-seizure medications such as carbamazepine, lamotrigine, levetiracetam, lorazepam, oxcarbazepine, phenobarbital, and phenytoin, medications to reduce ammonia levels (ammonia detoxicants), diet modification, intravenous fluids, dialysis, organ transplant, electrolytes, nutritional supplements, anti-inflammatories such as acetaminophen, ibuprofen, naproxen sodium and corticosteroids, anti-viral medications such as acyclovir, ganciclovir and goscarnet, anti-microbial medications, and surgery.

Exemplary treatments for various disorders and diseases is provided in the following:

| Disorder or disease | Exemplary treatments |
|---|---|
| pneumonia | Antivirals or antimicrobials |
| asthma | Bronchodilators (beta-2 agonists, anticholinergics); |
| | Corticosteroids; Leukotriene modifiers; Mast cell |
| | stabilizers; methylxanthines; immunomodulators |
| bronchitis | acetaminophen, hydration, possibly antitussives; |
| | mucolytics; inhaled beta-agonist |
| chronic obstructive | Stop smoking, inhaled bronchodilators, corticosteroids, |
| pulmonary disease (COPD) | supportive care such as oxygen therapy, pulmonary |
| | rehabilitation |
| rhabdomyolysis | intravascular expansion with IV fluids; mechanical |
| | ventilation; antimicrobials; discontinuation of inciting |
| | drugs |
| carnitine deficiency | L-carnitine supplements; dietary intervention; treatment of |
| | underlying condition such liver or kidney disease, |
| | malnutrition |
| carnitine palmitoyltransferase | Dietary intervention (minimize fats for energy) |
| 2 (CPT2) deficiency | |
| Reye's syndrome | Electrolytes and fluids, Insulin, corticosteroids, and |
| | diuretics |
| medium-chain acyl-CoA | Dietary intervention to prevent hypoglycemia episodes |
| dehydrogenase (MCAD) | |
| deficiency | |
| very-long-chain acyl-CoA | Dietary intervention to prevent hypoglycemia; use of low- |
| dehydrogenase (VLCAD) | fat nutritional supplements and medium-chain |
| deficiency | triglycerides |
| rotavirus infection | Prevention of dehydration, replenish electrolytes |
| hand-foot-mouth disease | Prevention of dehydration, topical oral anesthetic, |
| | acetaminophen or ibuprofen |
| viral infection | Antiviral medications, pain medication such as |
| | acetaminophen, ibuprofen or naproxen; prevention of |
| | dehydration, |
| influenza infection | Prevention of dehydration, decongestant, cough |
| | medication, Nonsteroidal anti-inflammatory drug, |
| | analgesic, antiviral |
| cyclic vomiting syndrome | Anti-nausea, analgesic, medications that suppress stomach |
| | acid, antidepressants, anti-seizure medications |
| jaundice, hyperbilirubinemia | In infants: Prevention of dehydration, phototherapy, |
| | exchange blood transfusion, intravenous immunoglobulin; |
| | In adults: treat underlying condition such as severe liver |
| | disease, hepatitis, bile duct obstructions |
| liver dysfunction | Stop alcohol use, lose weight, change to liver-friendly |
| | diet, antivirals, steroids, antibiotics, vitamins and |
| | supplements |
| epilepsy | Anti-seizure medication, brain surgery, MRI-guided |
| | stereotactic laser ablation, vagus nerve stimulation |
| West syndrome | adrenocorticotropic hormone (ACTH); Steroids, anti- |
| | seizure medications; adopt ketogenic diet |
| tonic-clonic seizures | Anti-seizure medication, brain surgery, MRI-guided |
| | stereotactic laser ablation, vagus nerve stimulation |
| febrile seizures | Diazepam, analgesic, non-steroidal anti-inflammatory |
| hypertrophic cardiomyopathy | beta-blockers, calcium channel blockers, avoid nitrates, |
| | diuretics, and angiotensin-converting enzyme (ACE) |
| | inhibitors; implantable cardioverter-defibrillator (ICD), |
| | surgical myectomy |
| abnormal ECG | Heart rate drugs, Pacemaker, catheter ablation, |
| mitral regurgitation (MR) | Low-sodium diets, surgery (mitral valve replacement, |
| | mitral valve repair) |
| myocarditis, | Corticosteroids, cardiac medications such as beta- |
| | blockers, ACE inhibitors, diuretic therapy |
| ventricular tachycardia, | Antiarrhythmics medications (such as sotalol, flecainide, |
| | propafenone, amiodarone), radiofrequency ablation, |
| | implantable cardioverter-defibrillator (ICD) |
| glutaric acidemia II (GAII) | Dietary intervention to prevent hypoglycemia, dietary |
| | supplementation with riboflavin, carnitine & other |
| | supplements |
| methylmalonic acidemia | cobalamin and carnitine supplements, a low-protein diet |
| | and avoidance of amino acids isoleucine, valine, threonine |
| | and methionine, vitamin B12 |
| respiratory syncytial virus | Acetaminophen, antiviral drug ribavirin, palivizumab, |
| (RSV) | bronchodilators, prevent dehydration |
| influenza A, influenza B | oseltamivir, zanamivir, peramivir, baloxavir, prevent |
| | dehydration, antipyretics |
| human herpes virus 6 (HHV- | Acetaminophen, maintain hydration, foscarnet, |
| 6) | ganciclovir, cidofovir, brincidofovir |
| Norovirus | Maintain hydration, anti-diarrheal |
| megalencephaly | Treat underlying cause, anti-seizure medications |
| hypoglycemia encephalopathy | Intravenous glucose, intramuscular glucagon, oral glucose |
| | gel |
| epileptic encephalopathy | Anti-seizure medications, vagal nerve stimulation, |
| | steroids, immunomodulatory therapies |
| hypoxic ischemic | Therapeutic hypothermia (TH), erythropoietin (EPO) |
| encephalopathy | |
| Leigh syndrome | Thiamine (vitamin B1) or thiamine derivatives, dietary |
| | intervention, anti-seizure medications, |
| periventricular leukomalacia | Physical therapy, occupational therapy, speech therapy |
| ifosfamide-induced | Intravenous methylene blue |
| encephalopathy | |
| acute focal bacterial nephritis | antimicrobials |
| (AFBN) encephalopathy | |
| leukoencephalopathy | Antiretroviral therapy, immune checkpoint inhibitors such |
| | as anti-PD-1 antibodies |

Based on the extensive knowledge in the art, the skilled person, physician, health care worker would know which drug, drug combination, therapeutic to administer to a patient in need thereof having one of the conditions, disorders or infections disclosed herein. The therapeutic or therapeutic combination would be known to the healthcare worker, as well as the amount needed for the patient, dosing requirements, and the like. The healthcare worker would obtain a pre-therapeutic administration GAG level and post-therapeutic GAG level in the patient in need thereof. One or more post-therapeutic GAG levels can be obtained for the patient depending on the condition, disorder or infection being treated. For example, once a viral infection has resolved, no further GAG level testing would be needed generally. However, for patients having, for example, a fatty acid disorder, then regular GAG testing would be needed to monitor the patient and the efficacy of the treatment being administered.

GAG level testing can be performed daily, weekly, more than once a week, every other week, ever 15 days, monthly, every other month, or at various intervals as necessary.

An elevated GAG level is any level at least one standard deviation or at least two standard deviations above the mean normal level for the indicated GAG. This level can be a single elevated GAG, two or more elevated GAGs, three or more elevated GAGs, four or more elevated GAGs, or five or more elevated GAGs. The presence of more than one elevated GAG at a level of at least one or more standard deviations above the mean is an indication of greater severity of the condition than if there is only one elevated GAG at one standard deviation away from the mean.

In certain embodiments, the subject is 0 to about 80 years old. In some embodiments, the subject is a newborn. In certain embodiments, the subject is 0 to about 2.9 years old. In some embodiments, the subject is aged from about 3 to about 4.9 years old. In some embodiments, the subject is aged from about 5 to about 9.9 years old. In some embodiments, the subject is aged from about 10 to about 14.9 years old. In some embodiments, the subject is aged from about 15 years old or older, such as about 15 to about 80 years old. In some embodiments, the subject is aged from about 15 to about 62 years old.

In certain embodiments, the control comprises an age-matched control.

Subject biological samples are collected for analysis from, for example, body fluids such as blood, plasma, serum, urine, cerebrospinal fluid (CSF), or other bodily fluids. No particular limitation is placed on the nature of the sample so long as it contains mucopolysaccharides. In certain embodiments, the biological sample is blood, plasma, serum, urine or tissue. In certain embodiments, the biological sample is a biological fluid selected from blood, plasma, serum, urine, and CSF. In certain embodiments, the biological sample is blood. In certain embodiments, the blood sample is a dried blood spot (DBS). In certain embodiments, the biological sample is CSF.

The sample may be subjected to filtration, including ultrafiltration, to concentrate mucopolysaccharides for analysis. No particular limitation is imposed on the filtration method and apparatus employed, so long as the filter does not allow mucopolysaccharides to pass there through but allows passage of molecules smaller than mucopolysaccharides in molecular weight. In one embodiment, the filter media is a 10K filter, e.g., Ultrafiltration Omega 10K membrane filter (PAL Life Sciences, NY). The membrane filters may be conveniently deployed in multiples format using, e.g., a multi-well plate (e.g., an AcroPrep™ 96-Well Filter Plate (PALL Life Sciences), to permit simultaneous processing of multiple samples. The filter plate may be optionally subjected to centrifugation, e.g., at 2,000×g for 15 minutes.

In certain embodiments, elevated GAGs can be measured by enzymatic digestion of the polysaccharide GAG chains into disaccharides and measuring the disaccharide levels of one or more disaccharides, using the method disclosed in the examples herein and methods known in the art. See, e.g., U.S. Pat. No. 9,982,288. The digesting enzyme may comprise, for example, chondroitinase B, chondroitinase C, or chondroitinase ABC. Chondroitinase ABC catalyzes the eliminative degradation of polysaccharides containing (1-4)-β-D-hexosaminyl and (1-3)-β-D-glucuronosyl or (1-3)-α-L-iduronosyl linkages to disaccharides containing 4-deoxy-β-D-gluc-4-enuronosyl groups. It acts on C4S, C6S, and DS, and acts slowly on hyaluronate. Other GAG-degrading enzymes that can be used for producing GAG degradation products include, for example, keratanase, keratanase II, heparitinase, heparitinase I, heparitinase II. A cocktail of enzymes, such as a cocktail of heparitinase, chondroitinase B, and keratanase, can be used. In certain embodiments, disaccharides measured are one or more of: ΔDiHS-NS: 2-deoxy-2-sulfamino-4-(4-deoxy-a-L-threo-hex-4-enopyranosyluronic acid)-D-glucose: ΔDiHS-0S: 2-acetamido-2-deoxy-4-O-(4-deoxy-a-L-threo-hex-4-enopyranosyluronic acid)-D-glucose: ΔDi-4S; DS: 2-acetamido-2-deoxy-4-O-(4-deoxy-a-L-threo-hex-4-enopyranosyluronic acid)-4-O-sulfo-D-glucose; Mono-S KS: mono-sulfated KS (Galβ1-4GlcNAc(6S)); and di-S KS: Di-sulfated KS (Gal(6S)β1-4GlcNAc(6S)). DS can be measured as Di-0S due to digestion of Di-4S to Di-0S by a 4S-sulfatase present in chondroitinase B.

The enzyme-digestion product is analyzed by an LC-MS/MS procedure. LC-MS/MS analysis of GAG-degradation products is described, for example, in US Pub. No. 2011/0008810. In the typical LC-MS/MS procedure, a liquid chromatography (LC) component separates sample components and then introduces them to a mass spectrometer (MS). The MS creates and detects charged ions. The LC/MS data may be used to provide information about the molecular weight, structure, identity, and quantity of specific sample components.

Mass spectrometers operate by converting the analyte molecules to a charged (ionized) state, with subsequent analysis of the ions and any fragment ions that are produced during the ionization process on the basis of their mass to charge ratio (m/z). A typical LC-MS/MS instrument contains: (i) an atmospheric pressure ionization source, typically an electrospray ionization source or an atmospheric pressure chemical ionization source, coupled by (ii) an ion-inlet and focusing component, which provides both transitions from atmospheric pressure to vacuum and ion-focusing, into (iii) a first mass-filtering device, which leads into (iv) a collision chamber that can be filled with low-pressure gas for collision-induced dissociation, followed by (v) a second mass-filtering device, and finally (vi) an ion-impact detector (electron multiplier). The construction, operation, and applications of LC-MS/MS instruments is reviewed by Grebe and Singh (2011), Clin. Biochem. Rev. 32: 5-31.

No particular limitation is imposed on the LC-MS/MS chromatography system, so long as the system can achieve adequate separation of disaccharides. Examples of a chromatography apparatus consist of the combination of a carbon graphite column and a reverse-phase HPLC column. Examples of commercially available carbon graphite columns include Hypercarb (2.0 mm i.d.×50 mm, 5 μm) (Thermo Electron Corp). Examples of reverse phase HPLC systems include the 1260 Infinity Quaternary LC System (Agilent Technologies, USA).

Representative chromatography conditions include, e.g., a column temperature of 50° C.; a mobile phase gradient elution of 5 mM ammonium acetate in acetonitrile-5 mM ammonium acetate buffer (pH 11.0); and a gradient condition program as follows: (i) initial composition of 0% acetonitrile kept for 0.1 min, linearly modified to 30% over 1.8 min, maintained at 30% for 0.3 min, modified to 0% over 0.01 min, and finally maintained at 0% for 2.5 min; flow rate: 0.7 milliliter per minute (ml/min). Further representative chromatography conditions include, e.g., mobile phases of 100 mM ammonia (A) and 100% acetonitrile. A gradient condition program can be as follows: the initial composition of 100% A is held for 1 minute, linearly modified to 30% B to 4 minutes, maintained at 30% B to 5.5 minutes, returned to 0% B at 6 minutes, and finally maintained at 0% B until 10 min; flow rate: 0.7 ml/min.

The mass spectroscopy component of the analysis may be carried out using any appropriate mass spectrometer, e.g., a 6460 Triple Quad mass spectrometer (Agilent Technologies) or equivalent device. The concentration of each disaccharide can be calculated by software, such as QQQ Quantitative Analysis software (Agilent Technologies).

### EXEMPLARY EMBODIMENTS

Among the embodiments provided herein are:
1. A method of treating a subject having a condition identified as having an elevated glycosaminoglycan (GAG) level comprising:
a) administering at least one therapeutic treatment to the subject;
b) monitoring the subject administered the therapeutic treatment for a reduction in a GAG level at least once to determine if the at least one therapeutic treatment reduces the GAG level;
c) optionally administering an additional therapeutic treatment to the subject;
   wherein the elevated GAG level is determined by enzymatic digestion of the GAG present in a biological sample to obtain disaccharides and determining the level of the disaccharides relative to normal.
2. The method of embodiment 1, wherein the disaccharide is selected from the group consisting of: 2-deoxy-2-sulfamino-4-(4-deoxy-a-L-threo-hex-4-enopyranosyluronic acid)-D-glucose (ΔDiHS-NS), 2-acetamido-2-deoxy-4-O-(4-deoxy-a-L-threo-hex-4-enopyranosyluronic acid)-D-glucose (ΔDiHS-0S), 2-acetamido-2-deoxy-4-O-(4-deoxy-a-L-threo-hex-4-enopyranosyluronic acid)-4-O-sulfo-D-glucose (ΔDi-4S; DS), mono-sulfated KS (Galβ1-4GlcNAc(6S)), and di-sulfated KS (Gal(6S)β1-4GlcNAc(6S)).
3. The method of embodiment 1 or 2, wherein the condition is selected from a respiratory condition, a renal disorder, a fatty acid metabolism disorder, a viral infection, a vomiting disorder, a liver disorder, epilepsy, hypoglycemia, myopathy, a developmental disorder, a hyperCKemia, a heart condition, acidosis, a viral encephalopathy, and a non-viral encephalopathy.
4. The method of any of embodiments 1 to 3, wherein the biological sample is a blood sample or cerebrospinal fluid (CSF).
5. A method of diagnosing severity of a condition severity in a subject in need thereof comprising:
a) measuring at least one GAG level that is elevated outside the normal range in a biological sample obtained from the subject; and
b) diagnosing condition severity as a patient having at least one GAG level two standard deviations above the mean of a control patient.
6. The method according to embodiment 5, wherein the biological sample is a body fluid selected from blood, plasma, serum, urine, and/or CSF.
7. The method, according to embodiment 5 or embodiment 6, further comprising administering at least one treatment for the condition severity diagnosed.
8. A method of treating a subject with a disease or disorder having an elevated glycosaminoglycan (GAG) level comprising:
a) assessing the level of at least one disaccharide obtained from a glycosaminoglycan in a biological sample containing glycosaminoglycans obtained from the subject that does not have a mucopolysaccharidosis (MPS);
b) determining the subject may be afflicted with a non-MPS disease or disorder when the level of the at least one disaccharide is elevated compared to the level of the same disaccharide in a biological sample from a control;
and
c) administering at least one treatment for the non-MPS disease or disorder to the subject.
9. The method according to embodiment 8, wherein the disease or disorder is at least one disease or disorder selected from the group consisting of: a respiratory condition, a renal disorder, a fatty acid metabolism disorder, a viral infection, a vomiting disorder, a liver disorder, epilepsy, hypoglycemia, myopathy, a developmental disorder, a hyperCKemia, a heart condition, acidosis, and encephalopathy.
10. The method according to embodiment 8, wherein the disease or disorder is at least one disease or disorder selected from the group consisting of: pneumonia, asthma, bronchitis, chronic obstructive pulmonary disease (COPD), rhabdomyolysis. carnitine deficiency, Reye's syndrome, carnitine palmitoyltransferase 2 (CPT2) deficiency, medium-chain acyl-CoA dehydrogenase (MCAD) deficiency, very-long-chain acyl-CoA dehydrogenase (VLCAD) deficiency, rotavirus infection, hand-foot-mouth disease viral infection, influenza infection, cyclic vomiting syndrome, jaundice, hyperbilirubinemia, liver dysfunction, epilepsy, West syndrome, tonic-clonic seizures, febrile seizures, hypertrophic cardiomyopathy, abnormal ECG, mitral regurgitation (MR), myocarditis, ventricular tachycardia, glutaric acidemia II (GAII), methylmalonic acidemia. respiratory syncytial virus (RSV), influenza A, influenza B, rotavirus, human herpes virus 6 (HHV-6), Norovirus, .megalencephaly, hypoglycemia encephalopathy, epileptic encephalopathy, hypoxic ischemic encephalopathy, Leigh syndrome, periventricular leukomalacia, ifosfamide-induced encephalopathy, acute focal bacterial nephritis (AFBN) encephalopathy, and leukoencephalopathy.
11. The method according to embodiment 8, wherein the disease or disorder is encephalopathy.
12. The method according to embodiment 9, wherein the disease or disorder is viral encephalopathy.
13. The method according to embodiment 12, wherein the at least one elevated disaccharide is selected from ΔDiHS-0S, ΔDi-4S, di-sulfated KS and the ratio of di-sulfated KS to total KS.
14. The method according to embodiment 12, wherein the disease or disorder is non-viral encephalopathy.
15. The method according to embodiment 14, wherein the at least one elevated disaccharide is selected from ΔDiHS-0S, ΔDiHS-NS, ΔDi-4S, mono-sulfated KS, di-sulfated KS, and the ratio of di-sulfated KS to total KS.
16. The method, according to any one of embodiments 9 to 15, wherein the control comprises an age-matched control.
17. The method, according to embodiment 8, wherein the disease or disorder is a bacterial infection.
18. The method, according to embodiment 17, wherein the bacterial infection is sepsis or meningitis.
19. The method, according to embodiment 17 or embodiment 18, wherein the at least one elevated disaccharide is ΔDiHS-0S.
20. The method, according to any one of embodiments 9 to 19, wherein the biological sample is a body fluid selected from blood, plasma, serum, urine and/or cerebrospinal fluid (CSF).

The practice of the disclosure will be more fully understood by reference to the following examples. The practice of the disclosure is illustrated by the following data. They should not, however, be construed as limiting the scope of the invention.

### Examples

### Example 1

In previous studies investigating GAG levels in blood or dried blood spots (DBS) of mucopolysaccharidoses (MPS) patients, elevated GAG levels were observed in a subset of control samples as well [7, 13]. Based on clinical findings and enzyme activity assay, it was determined that these control samples did not come from MPS patients. The following experiments were carried out to identify what kinds of diseases and conditions cause the elevation of GAGs, to determine which GAGs are elevated in each such disease and condition, and to explore whether the measurement of GAGs is a useful tool for prognosis, determination of disease stage, and monitoring therapeutic effect.

In this retrospective study, GAG levels and elevation patterns in blood in other diseases and conditions in childhood were investigated in dried blood spots of patients. The patients were grouped according to clinical diagnosis and condition. The clinical diagnosis groups include respiratory disorders, renal disorders, fatty acid metabolism disorders, viral infections, vomiting disorders, liver disorders, epilepsy, hypoglycemia, myopathy, developmental disorders, hyperCKemia, heart disease, acidosis, and encephalopathy.

### Materials and Methods

### Subjects

Serum samples were obtained with informed consent from 276 patients with various clinical conditions and diagnoses. Patient ages ranged from under 1 year of age to sixty-two years; however, only 14 patients were over 15 years old. Among the patients with various clinical conditions, 140 patients were 0-2.9 years old, 35 patients were 3-4.9 years old, 53 patients were 5-9.9 years old, 30 patients were 10-14.9 years old, and 18 patients were 15 years old or older.

The control group consisted of 44 patients with no findings or unique conditions. The mean and standard deviation for each GAG was found for each age group. If patients had GAG levels more or less than two standard deviations from the mean, then the patients were removed from the control group. The age range of control patients was zero to eighty years. Of these patients, 25 patients were 0-2.9 years old, 3 patients were 3-4.9 years old, 5 patients were 5-9.9 years old, 5 patients were 10-14.9 years old, and 6 patients were 15 years old or older.

Clinical diagnosis and enzyme activity assays confirmed that the patients did not have MPS. Twenty-two patients were clinically diagnosed with respiratory or renal conditions; 21 patients were diagnosed with some sort of fatty acid metabolism disorder; 7 patients were diagnosed with viral infections without encephalopathy or other symptoms; 13 patients were diagnosed with vomiting disorders; 18 patients were diagnosed with liver disorders; 33 patients were diagnosed with epilepsy; 22 patients were diagnosed with hypoglycemia; 12 patients were diagnosed with myopathy; 14 patients were diagnosed with developmental disorders; 12 patients were diagnosed with hyperCKemia; 15 patients were diagnosed with a heart condition; 16 patients were diagnosed with acidosis; 51 patients were diagnosed with viral encephalopathy, and 69 patients were diagnosed with non-viral encephalopathy. The total number of patients in these groups adds up to more than a total of 267 patients because some patients had overlapping conditions and were thus used in more than one group.

In addition, 198 dried blood spots (DBS) samples were collected from control newborns, including one MPS II patient, in a double-blind manner. Procedures were approved by the institutional review boards (IRBs) at Nemours and Alfred I. DuPont Hospital for Children (AIDHC) (approval number: 281498-21).

### Enzymes and Standards

Enzymes and stock solutions used to make standards were obtained from the Seikagaku Corporation. Heparitinase, chondroitinase B, and keratanase II were used to digest the polysaccharide GAG chains into their respective disaccharides: 2-deoxy-2-sulfamino-4-(4-deoxy-a-L-threo-hex-4-enopyranosyluronic acid)-D-glucose (ΔDiHS-NS), 2-acetamido-2-deoxy-4-O-(4-deoxy-a-L-threo-hex-4-enopyranosyluronic acid)-D-glucose (ΔDiHS-0S), 2-acetamido-2-deoxy-4-O-(4-deoxy-a-L-threo-hex-4-enopyranosyluronic acid)-4-O-sulfo-D-glucose (ΔDi-4S; DS), mono-sulfated KS (Galβ1-4GlcNAc(6S)), and di-sulfated KS (Gal(6S)β1-4GlcNAc(6S)). Stock solutions of the above disaccharides were used to make standard solutions by serial dilution consisting of 1000 ng/ml, 500 ng/ml, 250 ng/ml, 125 ng/ml, 62.5 ng/ml, 31.25 ng/ml, 15.625 ng/ml, and 7.8125 ng/ml of ΔDiHS-NS, ΔDiHS-0S, and ΔDi-4S, and 10,000 ng/ml, 5000 ng/ml, 2500 ng/ml, 1250 ng/ml, 625 ng/ml, 312.5 ng/ml, 156.25 ng/ml, and 78.125 ng/ml mono-sulfated KS (mono-S KS) and di-sulfated KS (di-S KS). Chondrosine was used as an internal standard.

### Sample Preparations

In AcroPrep™ Advance 96-Well Filter Plates with Ultrafiltration Omega 10K membrane filters (Pall Corporation, NY, USA), in order, the following was added: 40 microliters of a cocktail consisting of heparitinase, chondroitinase B, and keratanase; 90 microliters of 0.5 M Tris buffer at pH 7.0; 10 microliters of sample or standard; and 40 microliters of 0.5 M Tris buffer at pH 7.0. The filter plate was then incubated overnight on a 96-well receiver plate at 37° C. to allow digestion of the polysaccharides. The filter plate was then placed on a new receiver plate and centrifuged for 15 min at 2,500 rpm to filter the digested disaccharides. The processed samples were then injected into the liquid-chromatography tandem mass spectrometry (LC-MS/MS).

### LC-MS/MS:

The chromatographic system used has been described in earlier studies [13-18]. The mobile phases used were 100 mM ammonia (A) and 100% acetonitrile. The initial composition of 100% A was held for 1 minute, linearly modified to 30% B to 4 minutes, maintained at 30% B to 5.5 minutes, returned to 0% B at 6 minutes, and maintained at 0% B until 10 min. The flow rate was 0.7 milliliter per minute. DS was measured as Di-0S due to digestion of Di-4S to Di-0S by a 4S-sulfatase present in the chondroitinase B. The concentration of each disaccharide was calculated by QQQ Quantitative Analysis software.

### Statistical Analysis:

Patients were grouped according to diagnosis or condition. The control group consisted of patients with unique symptoms or no findings. Control patients with GAG levels more than two standard deviations above the mean were removed. Because GAG levels are also influenced by age, patients were then divided into the following age groups: X<3 years, 3≤X<5 years, 5≤X<10 years, 10≤X<15 years, and over 15 years of age. GAG levels were considered high if they were more than two standard deviations above the mean. The largest group comprised of patients with encephalopathy. This group was further divided by patients with viral or non-viral encephalopathy. The encephalopathy groups were compared to age-matched control groups with two-tailed t-tests. P-values less than 0.05 were considered significant.

### Results

Out of the 276 patients with various diseases or conditions, 147 patients (53.3%) had an elevation of at least one GAG. Of the patients with elevated GAGS, 46 patients (31.3%) had an elevation of just one GAG; 50 patients (34.0%) had an elevation of two GAGS; 16 patients (10.9%) had an elevation of three GAGS; 21 patients (14.3%) had an elevation of four GAGS; and 14 patients (9.5%) had an elevation of 5 GAGS. The data are tabulated in Table 1.

TABLE 1
Patients with Disease or Condition - GAGs elevated by two standard deviations
above the mean of control patients are in bold and italics

| | | | | | | | | Di- | Mono- | |
|---|---|---|---|---|---|---|---|---|---|---|
| | | | | Disease | ΔDiHS- | ΔDiHS- | | sulfated | sulfated | DiKS/ |
| Sex | Age | Diagnosis | Severity | Stage | 0S | NS | ΔDi4S | KS | KS | Total KS |
| M | 0.33 | rhabdomyolysis, | mild | initial | 21.5 | 6.4 | 110.5 | 22.2 | 421.1 | 5.0 |
| | | UTI | | | | | | | | |
| M | 0.33 | rhabdomyolysis, | mild | recovery | 16.6 | 8.2 | 96.9 | 108.4 | 420.3 | 20.5 |
| | | UTI | | | | | | | | |
| M | 0.0 | hypertrophic | moderate | screening | 25.7 | 21.6 | 152.3 | 59.2 | 549.5 | 9.7 |
| | | cardiomyopathy | | (stable) | | | | | | |
| M | 0 | left | severe | initial | 12.4 | 14.5 | 49.4 | 61.6 | 384.9 | 13.8 |
| | | megalocephaly | | | | | | | | |
| M | 0.0 | developmental | unknown | unknown | 36.5 | 18.2 | 104.0 | 147.7 | 912.1 | 13.9 |
| | | disorder | | | | | | | | |
| unknown | 0.04 | GAII | mild | screening | 56.1 | 16.7 | 113.8 | 260.6 | 1095.1 | 19.2 |
| | | suspect | | (stable) | | | | | | |
| M | 0.04 | jaundice, | mild | peak | 42.4 | 14.8 | 151.8 | 89.2 | 550.7 | 13.9 |
| | | metabolic | | | | | | | | |
| | | acidosis | | | | | | | | |
| M | 0.07 | ALTE, | moderate | recovery | 81.7 | 22.6 | 138.7 | 330.8 | 1254.2 | 20.9 |
| | | hypothermia, | | | | | | | | |
| | | hypoglycemia | | | | | | | | |
| M | 0.07 | hypoglycemic | severe | screening | 26.9 | 17.9 | 122.0 | 281.2 | 1015.0 | 21.7 |
| | | encephalopathy | | (stable) | | | | | | |
| F | 0.08 | acute | severe | initial | 30.3 | 0.8 | 46.8 | 80.4 | 513.2 | 13.5 |
| | | myocarditis | | | | | | | | |
| M | 0.08 | early | severe | peak | 16.8 | 2.0 | 23.6 | 117.0 | 252.8 | 31.6 |
| | | myoclonic | | | | | | | | |
| | | encephalopathy? | | | | | | | | |
| M | 0.08 | early | severe | peak | 27.3 | 1.7 | 121.2 | 97.7 | 838.4 | 10.4 |
| | | myoclonic | | | | | | | | |
| | | encephalopathy? | | | | | | | | |
| F | 0.08 | epilepsy, | unknown | screening | 25.2 | 25.3 | 101.8 | 162.8 | 955.7 | 14.6 |
| | | seizure | | (stable) | | | | | | |
| F | 0.17 | acute | severe | initial | 65.1 | 131.0 | 542.6 | 232.7 | 699.4 | 25.0 |
| | | encephalopathy | | | | | | | | |
| F | 0.2 | encephalopathy, | severe | initial | 38.1 | 37.5 | 236.1 | 458.6 | 1458.3 | 23.9 |
| | | RSV+, | | | | | | | | |
| | | hepatitis | | | | | | | | |
| F | 0.17 | secondary | mild | screening | 31.3 | 19.7 | 114.5 | 326.1 | 1002.1 | 24.6 |
| | | carnitine | | (stable) | | | | | | |
| | | deficiency | | | | | | | | |
| F | 0.25 | afebrile | moderate | screening | 30.9 | 24.0 | 120.3 | 153.7 | 918.1 | 14.3 |
| | | seizure, | | (stable) | | | | | | |
| | | epilepsy? | | | | | | | | |
| F | 0.25 | epilepsy | moderate | peak | 4.6 | 7.1 | 45.3 | 129.1 | 637.3 | 16.8 |
| F | 0.25 | coarctation | moderate | screening | 25.2 | 10.8 | 68.2 | 92.5 | 584.6 | 13.7 |
| | | of the | | (stable) | | | | | | |
| | | aorta, lactic | | | | | | | | |
| | | acidemia, | | | | | | | | |
| | | CK | | | | | | | | |
| | | elevation | | | | | | | | |
| F | 0.3 | influenza | unknown | unknown | 112.9 | 353.1 | 928.5 | 630.1 | 1193.0 | 34.6 |
| | | encephalopathy | | | | | | | | |
| F | 0.3 | influenza | unknown | unknown | 68.5 | 158.0 | 444.9 | 483.6 | 945.8 | 33.8 |
| | | encephalopathy | | | | | | | | |
| F | 0.25 | encephalopathy | severe | initial | 44.5 | 62.2 | 296.6 | 240.9 | 818.9 | 22.7 |
| M | 0.25 | acute | moderate | initial | 60.8 | 210.1 | 495.2 | 336.8 | 554.3 | 37.8 |
| | | encephalopathy, | | | | | | | | |
| | | DIC | | | | | | | | |
| M | 0.33 | acute | severe | initial | 28.3 | 53.8 | 237.6 | 330.2 | 967.2 | 25.5 |
| | | encephalopathy | | | | | | | | |
| M | 0.33 | encephalopathy?, | severe | unknown | 19.0 | 17.4 | 96.6 | 94.4 | 524.2 | 15.3 |
| | | lactic | | | | | | | | |
| | | acidemia | | | | | | | | |
| F | 0.3 | CPT2 | moderate | peak | 31.3 | 20.5 | 163.3 | 194.1 | 1109.8 | 14.9 |
| | | deficiency | | | | | | | | |
| F | 0.33 | West | moderate | screening | 24.2 | 20.1 | 66.1 | 78.8 | 570.5 | 12.1 |
| | | syndrome | | (stable) | | | | | | |
| F | 0.33 | shock, | severe | peak | 30.0 | 59.8 | 267.0 | 187.9 | 519.4 | 26.6 |
| | | encephalopathy, | | | | | | | | |
| | | Reye | | | | | | | | |
| | | syndrome?, | | | | | | | | |
| | | CPT2 | | | | | | | | |
| | | deficiency | | | | | | | | |
| F | 0.42 | hyperbilirubinemia, | moderate | screening | 32.3 | 16.2 | 130.8 | 140.9 | 674.3 | 17.3 |
| | | transient | | (stable) | | | | | | |
| | | tachypnea | | | | | | | | |
| F | 0.42 | West | moderate | screening | 35.2 | 16.2 | 137.6 | 312.8 | 1165.2 | 21.2 |
| | | syndrome | | (stable) | | | | | | |
| unknown | 0.42 | seizure | moderate | screening | 13.4 | 18.2 | 121.7 | 117.5 | 762.0 | 13.4 |
| | | | | (stable) | | | | | | |
| F | 0.42 | partial | moderate | screening | 16.5 | 51.1 | 56.4 | 98.6 | 488.2 | 16.8 |
| | | convulsion | | (stable) | | | | | | |
| F | 0.42 | muscle | mild | screening | 15.4 | 24.8 | 127.2 | 81.1 | 662.9 | 10.9 |
| | | weakness, | | (stable) | | | | | | |
| | | low-oxygen | | | | | | | | |
| | | ischemic | | | | | | | | |
| | | injury? | | | | | | | | |
| M | 0.42 | SIDS?, | moderate | initial | 20.7 | 37.6 | 42.4 | 126.0 | 725.2 | 14.8 |
| | | metabolic | | | | | | | | |
| | | acidosis | | | | | | | | |
| M | 0.4 | GAII | unknown | screening | 26.5 | 12.4 | 96.6 | 314.1 | 741.9 | 29.7 |
| | | suspect | | (stable) | | | | | | |
| unknown | 0.42 | GAII | unknown | screening | 21.7 | 12.4 | 114.4 | 85.8 | 789.3 | 9.8 |
| | | | | (stable) | | | | | | |
| M | 0.42 | metabolic | mild | screening | 26.5 | 5.7 | 44.0 | 87.3 | 544.0 | 13.8 |
| | | acidosis | | (stable) | | | | | | |
| M | 0.42 | hypoxic | severe | peak | 1.6 | 0.8 | 57.8 | 15.1 | 266.4 | 5.3 |
| | | ischemic | | | | | | | | |
| | | encephalopathy | | | | | | | | |
| M | 0.42 | acute | severe | initial | 30.6 | 33.4 | 188.7 | 216.3 | 769.4 | 21.9 |
| | | encephalopathy | | | | | | | | |
| F | 0.42 | acute | moderate | initial | 30.3 | 19.8 | 148.8 | 338.8 | 909.6 | 27.1 |
| | | encephalopathy | | | | | | | | |
| | | with | | | | | | | | |
| | | biphasic | | | | | | | | |
| | | seizures | | | | | | | | |
| | | and late | | | | | | | | |
| | | reduced | | | | | | | | |
| | | diffusion | | | | | | | | |
| | | (suspect) | | | | | | | | |
| M | 0.42 | acute | moderate | initial | 20.7 | 9.8 | 134.6 | 148.7 | 687.4 | 17.8 |
| | | encephalopathy | | | | | | | | |
| | | suspect | | | | | | | | |
| M | 0.5 | vomiting | mild | screening | 23.4 | 11.8 | 81.8 | 199.3 | 550.5 | 26.6 |
| | | | | (stable) | | | | | | |
| M | 0.5 | TAM, | mild | screening | 16.7 | 13.7 | 100.4 | 77.9 | 488.4 | 13.8 |
| | | elevation | | (stable) | | | | | | |
| | | of AST, ALT | | | | | | | | |
| M | 0.5 | epilepsy, | mild | screening | 19.3 | 24.1 | 137.0 | 171.8 | 846.1 | 16.9 |
| | | seizure | | (stable) | | | | | | |
| F | 0.5 | acute | severe | peak | 61.2 | 393.1 | 1128.9 | 235.7 | 592.0 | 28.5 |
| | | encephalopathy | | | | | | | | |
| M | 0.5 | hemorrhagic | severe | initial | 9.5 | 20.9 | 117.3 | 107.6 | 454.8 | 19.1 |
| | | shock and | | | | | | | | |
| | | encephalopathy | | | | | | | | |
| | | syndrome | | | | | | | | |
| F | 0.58 | developmental | mild | screening | 19.7 | 7.9 | 62.6 | 172.6 | 476.4 | 26.6 |
| | | delay, | | (stable) | | | | | | |
| | | renal | | | | | | | | |
| | | failure | | | | | | | | |
| M | 0.58 | acute | moderate | screening | 17.5 | 11.0 | 99.8 | 242.4 | 750.9 | 24.4 |
| | | encephalopathy | | (stable) | | | | | | |
| F | 0.58 | acute | moderate | recovery | 30.2 | 13.9 | 131.0 | 152.7 | 603.9 | 20.2 |
| | | encephalopathy | | | | | | | | |
| F | 0.58 | acute | severe | peak | 58.9 | 97.0 | 418.3 | 239.3 | 874.6 | 21.5 |
| | | encephalopathy | | | | | | | | |
| F | 0.58 | floppy | mild | screening | 11.6 | 17.2 | 88.5 | 110.7 | 675.8 | 14.1 |
| | | infant | | (stable) | | | | | | |
| F | 0.67 | muscle | mild | screening | 24.7 | 16.3 | 101.2 | 98.3 | 782.6 | 11.2 |
| | | weakness | | (stable) | | | | | | |
| M | 0.67 | hyper-CK- | mild | screening | 11.6 | 12.5 | 80.7 | 108.7 | 461.2 | 19.1 |
| | | emia, | | (stable) | | | | | | |
| | | pneumonia | | | | | | | | |
| | | developmental | | | | | | | | |
| | | delay, | | | | | | | | |
| | | hypotonic | | | | | | | | |
| M | 0.67 | Acute | severe | initial | 70.5 | 253.2 | 782.8 | 175.1 | 557.9 | 23.9 |
| | | encephalopathy | | | | | | | | |
| M | 0.7 | post ALTE, | severe | peak | 27.2 | 25.2 | 107.4 | 82.0 | 553.1 | 12.9 |
| | | cardiac | | | | | | | | |
| | | arrest, | | | | | | | | |
| | | brain | | | | | | | | |
| | | edema, on | | | | | | | | |
| | | hypothermia | | | | | | | | |
| | | therapy | | | | | | | | |
| F | 0.75 | rotavirus | unknown | unknown | 27.8 | 20.7 | 123.9 | 192.9 | 841.1 | 18.7 |
| | | gastroenteritis, | | | | | | | | |
| | | generalized | | | | | | | | |
| | | seizure | | | | | | | | |
| M | 0.8 | AGE | moderate | peak | 19.4 | 7.3 | 71.6 | 144.7 | 508.8 | 22.1 |
| | | dehydration, | | | | | | | | |
| | | liver | | | | | | | | |
| | | dysfunction | | | | | | | | |
| M | 0.75 | jaundice | mild | screening | 19.4 | 10.8 | 73.6 | 259.7 | 941.5 | 21.6 |
| | | | | (stable) | | | | | | |
| F | 0.75 | development | mild | screening | 27.4 | 13.1 | 82.4 | 144.8 | 452.8 | 24.2 |
| | | disorders, | | (stable) | | | | | | |
| | | hypoglycemia | | | | | | | | |
| M | 0.8 | MR | mild | screening | 21.5 | 10.7 | 79.4 | 111.1 | 607.8 | 15.5 |
| | | | | (stable) | | | | | | |
| F | 0.75 | acute | moderate | initial | 42.5 | 46.8 | 243.5 | 344.2 | 867.8 | 28.4 |
| | | encephalopathy | | | | | | | | |
| F | 0.75 | Leigh | moderate | peak | 12.5 | 10.1 | 50.7 | 94.7 | 483.6 | 16.4 |
| | | encephalopathy | | | | | | | | |
| | | suspect | | | | | | | | |
| M | 0.75 | mitochondrial | severe | peak | 23.6 | 19.2 | 123.6 | 111.1 | 572.7 | 16.2 |
| | | disease | | | | | | | | |
| | | suspect, | | | | | | | | |
| | | Leigh | | | | | | | | |
| | | encephalopathy | | | | | | | | |
| F | 0.8 | norovirus | severe | peak | 61.4 | 54.5 | 249.3 | 305.3 | 630.8 | 32.6 |
| | | encephalopathy, | | | | | | | | |
| | | artificial | | | | | | | | |
| | | respiration+ | | | | | | | | |
| F | 0.83 | developmental | mild | screening | 35.7 | 44.9 | 49.5 | 93.9 | 607.1 | 13.4 |
| | | disorder | | (stable) | | | | | | |
| F | 0.83 | developmental | mild | screening | 21.0 | 11.8 | 75.5 | 124.3 | 942.1 | 11.7 |
| | | disorder | | (stable) | | | | | | |
| M | 0.83 | methylmalonic | moderate | screening | 22.9 | 11.9 | 86.0 | 63.1 | 396.7 | 13.7 |
| | | acidemia | | (stable) | | | | | | |
| M | 0.83 | High | unknown | screening | 17.0 | 8.1 | 62.0 | 138.2 | 769.6 | 15.2 |
| | | CKemia | | (stable) | | | | | | |
| M | 0.8 | fatty acid | unknown | screening | 18.5 | 9.0 | 63.8 | 118.5 | 931.4 | 11.3 |
| | | metabolism | | (stable) | | | | | | |
| | | disorder | | | | | | | | |
| | | suspect | | | | | | | | |
| M | 0.92 | epilepsy | mild | screening | 12.1 | 8.4 | 65.3 | 88.4 | 551.6 | 13.8 |
| | | | | (stable) | | | | | | |
| M | 0.92 | developmental | mild | screening | 6.8 | 6.5 | 49.3 | 73.4 | 324.0 | 18.5 |
| | | delay, | | (stable) | | | | | | |
| | | hypopituitary | | | | | | | | |
| | | gland | | | | | | | | |
| M | 0.92 | West | moderate | screening | 46.7 | 20.1 | 173.2 | 363.5 | 818.0 | 30.8 |
| | | syndrome | | (stable) | | | | | | |
| M | 1 | myopathy, | mild | screening | 21.6 | 9.4 | 72.7 | 133.3 | 892.4 | 13.0 |
| | | rhabdomyolysis? | | (stable) | | | | | | |
| F | 1.0 | secondary | moderate | peak | 23.3 | 16.6 | 102.2 | 189.7 | 1190.9 | 13.7 |
| | | carnitine | | | | | | | | |
| | | deficiency, | | | | | | | | |
| | | CFPN-PI | | | | | | | | |
| | | induced | | | | | | | | |
| F | 1.0 | MCAD | moderate | initial | 6.0 | 9.7 | 58.1 | 54.7 | 319.1 | 14.6 |
| M | 1.0 | fatty acid | mild | screening | 22.9 | 4.8 | 72.1 | 132.8 | 786.2 | 14.4 |
| | | metabolism | | (stable) | | | | | | |
| | | disorder | | | | | | | | |
| | | suspect | | | | | | | | |
| M | 1.0 | CPT2 | moderate | screening | 12.2 | 7.4 | 57.4 | 198.6 | 515.9 | 27.8 |
| | | deficiency | | (stable) | | | | | | |
| M | 1 | secondary | mild | screening | 20.6 | 14.0 | 79.7 | 134.0 | 878.5 | 13.2 |
| | | carnitine | | (stable) | | | | | | |
| | | deficiency, | | | | | | | | |
| | | mental | | | | | | | | |
| | | deficiency, | | | | | | | | |
| | | epilepsy | | | | | | | | |
| F | 1.00 | inguinal | mild | screening | 22.7 | 4.7 | 23.6 | 102.8 | 634.5 | 13.9 |
| | | hernia, | | (stable) | | | | | | |
| | | liver | | | | | | | | |
| | | disorder | | | | | | | | |
| F | 1 | hypoglycemia, | moderate | recovery | 23.3 | 21.6 | 66.0 | 100.9 | 617.5 | 14.0 |
| | | congenital | | | | | | | | |
| | | biliary | | | | | | | | |
| | | atresia | | | | | | | | |
| M | 1 | a bilious | moderate | recovery | 9.1 | 24.2 | 89.9 | 183.1 | 1229.5 | 13.0 |
| | | attack, | | | | | | | | |
| | | disturbance | | | | | | | | |
| | | of | | | | | | | | |
| | | consciousness, | | | | | | | | |
| | | fatty | | | | | | | | |
| | | liver | | | | | | | | |
| F | 1 | afebrile | mild | screening | 14.1 | 2.9 | 75.4 | 138.4 | 871.1 | 13.7 |
| | | seizure, | | (stable) | | | | | | |
| | | epilepsy? | | | | | | | | |
| F | 1 | afebrile | moderate | screening | 27.9 | 22.6 | 93.9 | 94.0 | 605.9 | 13.4 |
| | | convulsion, | | (stable) | | | | | | |
| | | hypoglycemia | | | | | | | | |
| M | 1.0 | tonic-clonic | mild | screening | 16.3 | 8.8 | 68.0 | 100.7 | 576.9 | 14.9 |
| | | seizure | | (stable) | | | | | | |
| M | 1 | ketotic | moderate | recovery | 33.4 | 11.2 | 99.9 | 139.0 | 840.2 | 14.2 |
| | | hypoglycemia | | | | | | | | |
| M | 1.00 | ketotic | moderate | initial | 16.3 | 9.7 | 84.5 | 199.7 | 558.9 | 26.3 |
| | | hypoglycemia | | | | | | | | |
| M | 1.00 | low glucose | mild | screening | 20.5 | 6.1 | 28.4 | 68.7 | 407.7 | 14.4 |
| | | | | (stable) | | | | | | |
| F | 1 | hyper-CK- | mild | screening | 13.1 | 9.9 | 60.6 | 157.7 | 717.1 | 18.0 |
| | | emia, | | (stable) | | | | | | |
| | | muscular | | | | | | | | |
| | | dystrophy? | | | | | | | | |
| unknown | 1 | metabolic | mild | screening | 22.8 | 11.3 | 84.3 | 83.8 | 632.0 | 11.7 |
| | | myopathy | | (stable) | | | | | | |
| | | suspect | | | | | | | | |
| F | 1.0 | mental and | mild | screening | 22.5 | 12.7 | 61.2 | 125.5 | 830.3 | 13.1 |
| | | development | | (stable) | | | | | | |
| | | disorder | | | | | | | | |
| F | 1.0 | developmental | mild | screening | 19.8 | 13.3 | 80.2 | 120.5 | 798.1 | 13.1 |
| | | disorder, | | (stable) | | | | | | |
| | | mitochondria | | | | | | | | |
| | | disorder? | | | | | | | | |
| M | 1.0 | developmental | mild | screening | 25.3 | 30.0 | 188.7 | 121.8 | 1071.7 | 10.2 |
| | | delay | | (stable) | | | | | | |
| M | 1.0 | developmental | mild | screening | 23.0 | 10.6 | 62.7 | 165.7 | 1095.1 | 13.1 |
| | | disorder, | | (stable) | | | | | | |
| | | hyperlactatemia | | | | | | | | |
| M | 1.00 | developmental | mild | screening | 8.9 | 4.6 | 31.4 | 84.9 | 967.9 | 8.1 |
| | | disorders | | (stable) | | | | | | |
| F | 1 | development | mild | screening | 24.0 | 1.0 | 84.5 | 129.8 | 723.3 | 15.2 |
| | | disorder, | | (stable) | | | | | | |
| | | febrile | | | | | | | | |
| | | delirium, | | | | | | | | |
| | | metabolic | | | | | | | | |
| | | disorders? | | | | | | | | |
| F | 1 | hyper-CK- | mild | screening | 21.3 | 1.6 | 43.9 | 112.4 | 597.0 | 15.8 |
| | | emia | | (stable) | | | | | | |
| M | 1 | rotavirus | severe | screening | 5.4 | 5.3 | 58.9 | 208.7 | 749.8 | 21.8 |
| | | encephalopathy, | | (stable) | | | | | | |
| | | encephalitis, | | | | | | | | |
| | | myocarditis | | | | | | | | |
| M | 1.0 | MR, | mild | screening | 20.4 | 9.2 | 66.6 | 115.4 | 828.3 | 12.2 |
| | | microcephaly, | | (stable) | | | | | | |
| | | hyperlactatemia | | | | | | | | |
| unknown | 1.0 | head | mild | screening | 22.0 | 3.3 | 64.2 | 334.4 | 988.8 | 25.3 |
| | | circumference | | (stable) | | | | | | |
| | | enlargement, | | | | | | | | |
| | | MR | | | | | | | | |
| M | 1.00 | metabolic | moderate | initial | 15.1 | 5.6 | 40.8 | 48.7 | 334.2 | 12.7 |
| | | acidosis | | | | | | | | |
| M | 1 | SIDS-like, | severe | initial | 46.2 | 2828.8 | 12475.3 | 285.1 | 768.0 | 27.1 |
| | | influ A+, | | | | | | | | |
| | | acidosis, | | | | | | | | |
| | | hyperlactatemia | | | | | | | | |
| M | 1 | developmental | mild | screening | 20.8 | 12.2 | 82.6 | 139.0 | 857.6 | 13.9 |
| | | delay, | | (stable) | | | | | | |
| | | rota virus | | | | | | | | |
| F | 1.0 | HHV-6 | severe | peak | 48.8 | 64.0 | 309.2 | 260.3 | 854.8 | 23.3 |
| | | encephalopathy | | | | | | | | |
| F | 1.0 | Virus acute | severe | peak | 84.6 | 109.6 | 403.1 | 433.7 | 1150.2 | 27.4 |
| | | encephalopathy | | | | | | | | |
| F | 1.0 | Exanthema | severe | initial | 42.2 | 67.5 | 345.3 | 353.5 | 1061.3 | 25.0 |
| | | subitem | | | | | | | | |
| | | encephalopathy | | | | | | | | |
| M | 1.0 | HHV6 | moderate | initial | 32.8 | 16.7 | 148.2 | 166.0 | 714.2 | 18.9 |
| | | encephalopathy | | | | | | | | |
| | | suspect | | | | | | | | |
| M | 1 | acute | moderate | unknown | 17.6 | 3.9 | 90.9 | 121.1 | 724.8 | 14.3 |
| | | encephalopathy | | | | | | | | |
| | | with | | | | | | | | |
| | | biphasic | | | | | | | | |
| | | seizures | | | | | | | | |
| | | and late | | | | | | | | |
| | | reduced | | | | | | | | |
| | | diffusion, | | | | | | | | |
| | | HHV6 | | | | | | | | |
| F | 1 | influenza | moderate | peak | 28.5 | 10.0 | 56.1 | 312.1 | 825.2 | 27.4 |
| | | encephalopathy | | | | | | | | |
| | | (A+ | | | | | | | | |
| | | suspect) | | | | | | | | |
| M | 1 | acute | moderate | recovery | 30.6 | 49.6 | 134.3 | 191.6 | 663.1 | 22.4 |
| | | encephalopathy | | | | | | | | |
| M | 1 | hypoxic | moderate | recovery | 24.0 | 4.4 | 57.1 | 212.5 | 389.7 | 35.3 |
| | | ischemic | | | | | | | | |
| | | encephalopathy | | | | | | | | |
| M | 1 | acute | moderate | initial | 30.8 | 1.2 | 47.0 | 180.2 | 451.9 | 28.5 |
| | | encephalopathy | | | | | | | | |
| F | 1 | acute | moderate | screening | 45.9 | 12.0 | 116.1 | 321.4 | 1031.7 | 23.8 |
| | | encephalopathy | | (stable) | | | | | | |
| M | 1.0 | acute | severe | initial | 20.4 | 2.4 | 83.4 | 54.6 | 489.4 | 10.0 |
| | | encephalopathy | | | | | | | | |
| | | with | | | | | | | | |
| | | biphasic | | | | | | | | |
| | | seizures | | | | | | | | |
| | | and late | | | | | | | | |
| | | reduced | | | | | | | | |
| | | diffusion | | | | | | | | |
| F | 1 | acute | unknown | initial | 25.2 | 11.9 | 84.1 | 488.2 | 1124.3 | 30.3 |
| | | encephalopathy | | | | | | | | |
| | | suspect | | | | | | | | |
| F | 2 | periventricular | mild | initial | 21.0 | 27.8 | 157.6 | 225.9 | 861.9 | 20.8 |
| | | leukomalacia, | | | | | | | | |
| | | cerebral | | | | | | | | |
| | | palsy, | | | | | | | | |
| | | rhabdomyolysis | | | | | | | | |
| M | 2 | artificial | severe | peak | 11.1 | 13 | 96 | 149.8 | 424.3 | 26.1 |
| | | respiration, | | | | | | | | |
| | | pulmonary | | | | | | | | |
| | | hypertensi | | | | | | | | |
| | | on crisis | | | | | | | | |
| M | 2 | asthma, | mild | screening | 17.5 | 7.9 | 36.1 | 134.6 | 719.1 | 15.8 |
| | | lactacidemia | | (stable) | | | | | | |
| unkn | 2.00 | asthma, | mild | screening | 16.2 | 6.4 | 39.9 | 184.2 | 1198.7 | 13.3 |
| own | | lactacidemia | | (stable) | | | | | | |
| F | 2.0 | VLCAD | moderat | recovery | 8.3 | 8.4 | 48.5 | 162.7 | 929.7 | 14.9 |
| | | suspected, | e | | | | | | | |
| | | repeated | | | | | | | | |
| | | hypoglycemia | | | | | | | | |
| F | 2.0 | carnitine | mild | screening | 5.8 | 5.9 | 60.9 | 128.0 | 746.9 | 14.6 |
| | | deficiency | | (stable) | | | | | | |
| | | suspect | | | | | | | | |
| F | 2 | shock, | moderate | peak | 95.1 | 303.5 | 1022.1 | 263.2 | 850.3 | 23.6 |
| | | congenital | | | | | | | | |
| | | heart | | | | | | | | |
| | | disease | | | | | | | | |
| | | (PA, VSD | | | | | | | | |
| | | post op.), | | | | | | | | |
| | | carnitine | | | | | | | | |
| | | deficiency, | | | | | | | | |
| | | hypoglycemia | | | | | | | | |
| F | 2 | ketotic | mild | initial | 17.6 | 0.5 | 21.2 | 113.1 | 612.3 | 15.6 |
| | | hypoglycemia, | | | | | | | | |
| | | vomiting | | | | | | | | |
| M | 2 | liver | moderate | recovery | 35.5 | 12.3 | 89.2 | 81.8 | 821.5 | 9.1 |
| | | dysfunction | | | | | | | | |
| F | 2 | severe | severe | recovery | 21.8 | 13.9 | 61.8 | 85.3 | 393.6 | 17.8 |
| | | myoclonic | | | | | | | | |
| | | epilepsy | | | | | | | | |
| M | 2 | left-handed | mild | screening | 11.2 | 10.2 | 72.0 | 178.1 | 1044.2 | 14.6 |
| | | convulsion | | (stable) | | | | | | |
| F | 2 | convulsion, | moderate | recovery | 28.0 | 9.0 | 65.8 | 380.7 | 1060.2 | 26.4 |
| | | hypoglycemia | | | | | | | | |
| M | 2 | convulsive | severe | peak | 34.3 | 16.6 | 130.6 | 174.9 | 1156.4 | 13.1 |
| | | seizure | | | | | | | | |
| M | 2 | febrile | mild | recovery | 24.0 | 15.4 | 58.3 | 201.5 | 917.2 | 18.0 |
| | | convulsion | | | | | | | | |
| F | 2 | convulsive | severe | recovery | 33.6 | 18.8 | 110.8 | 229.8 | 736.3 | 23.8 |
| | | seizure | | | | | | | | |
| M | 2 | hypoglycemic | moderate | screening | 12.3 | 7.6 | 36.8 | 195.3 | 1229.8 | 13.7 |
| | | attack | | (stable) | | | | | | |
| F | 2 | MR | mild | screening | 17.8 | 8.1 | 77.8 | 143.8 | 670.4 | 17.7 |
| | | | | (stable) | | | | | | |
| F | 2 | HHV-6 | mild | screening | 25.4 | 5.3 | 42.9 | 73.7 | 659.1 | 10.1 |
| | | encephalopathy | | (stable) | | | | | | |
| F | 2 | viral | moderate | recovery | 12.9 | 7.4 | 40.1 | 87.5 | 375.4 | 18.9 |
| | | encephalopathy, | | | | | | | | |
| | | influenza | | | | | | | | |
| M | 2.0 | encephalopathy, | severe | peak | 15.6 | 18.9 | 101.4 | 174.3 | 1201.3 | 12.7 |
| | | unknown | | | | | | | | |
| | | virus- | | | | | | | | |
| | | related | | | | | | | | |
| M | 2 | acute | moderate | peak | 15.1 | 14.7 | 92.6 | 257.3 | 917.4 | 21.9 |
| | | encephalopathy | | | | | | | | |
| M | 2 | acute | severe | recovery | 36.1 | 0.7 | 71.9 | 172.0 | 509.1 | 25.3 |
| | | encephalopathy | | | | | | | | |
| M | 2 | acute | moderate | screening | 26.2 | 18.8 | 98.4 | 120.0 | 768.2 | 13.5 |
| | | encephalopathy | | (stable) | | | | | | |
| F | 2 | Leigh | mild | peak | 18.6 | 1.4 | 64.5 | 124.7 | 448.7 | 21.7 |
| | | encephalopathy | | | | | | | | |
| F | 2 | epilepsy, | severe | peak | 3.4 | 9.3 | 79.8 | 130.4 | 579.6 | 18.4 |
| | | acute | | | | | | | | |
| | | encephalopathy | | | | | | | | |
| | | with | | | | | | | | |
| | | biphasic | | | | | | | | |
| | | seizures | | | | | | | | |
| | | and late | | | | | | | | |
| | | reduced | | | | | | | | |
| | | diffusion | | | | | | | | |
| M | 2.0 | secondary | moderate | recovery | 20.4 | 10.5 | 68.3 | 448.9 | 1263.7 | 26.2 |
| | | carnitine | | | | | | | | |
| | | deficiency, | | | | | | | | |
| | | post | | | | | | | | |
| | | hypoglycemia | | | | | | | | |
| | | (glu 16 | | | | | | | | |
| | | mg/dl) | | | | | | | | |
| F | 3 | rhabdomyolysis, | moderate | recovery | 18.3 | 12.3 | 63.0 | 123.8 | 817.8 | 13.1 |
| | | Hypernatremia, | | | | | | | | |
| | | Adeno, | | | | | | | | |
| | | Rota+ | | | | | | | | |
| M | 3.0 | carnitine | mild | recovery | 32.9 | 19.8 | 115.0 | 88.5 | 419.8 | 17.4 |
| | | deficiency | | | | | | | | |
| F | 3.0 | hypocarnitinemia, | mild | screening | 24.6 | 9.0 | 69.2 | 134.4 | 901.6 | 13.0 |
| | | under | | (stable) | | | | | | |
| | | carnitine | | | | | | | | |
| | | administration | | | | | | | | |
| M | 3.0 | cyclic | moderate | recovery | 16.9 | 7.7 | 54.9 | 133.3 | 896.9 | 12.9 |
| | | vomiting | | | | | | | | |
| F | 3.00 | afebrile | mild | screening | 13.5 | 19.2 | 24.5 | 126.2 | 604.0 | 17.3 |
| | | convulsion | | (stable) | | | | | | |
| F | 3 | hypoglycemia | mild | screening | 18.1 | 4.0 | 58.9 | 121.4 | 787.9 | 13.4 |
| | | attack | | (stable) | | | | | | |
| F | 3 | Low | mild | screening | 30.7 | 5.8 | 99.0 | 220.9 | 1149.3 | 16.1 |
| | | glucose | | (stable) | | | | | | |
| F | 3.0 | ketotic | moderate | screening | 32.9 | 8.6 | 29.9 | 118.6 | 612.3 | 16.2 |
| | | hypoglycemia, | | (stable) | | | | | | |
| | | impaired | | | | | | | | |
| | | consciousness | | | | | | | | |
| M | 3 | rotavirus | moderate | recovery | 13.0 | 12.5 | 57.9 | 118.0 | 660.4 | 15.2 |
| | | encephalopathy | | | | | | | | |
| M | 3 | rotavirus | moderate | recovery | 0.8 | 0.5 | 3.1 | 75.5 | 659.4 | 10.3 |
| | | encephalopathy | | | | | | | | |
| M | 3.0 | encephalopathy | severe | recovery | 1.5 | 5.6 | 58.8 | 104.0 | 626.5 | 14.2 |
| | | (virus | | | | | | | | |
| | | unknown) | | | | | | | | |
| M | 3 | acute | severe | recovery | 7.8 | 5.4 | 64.9 | 104.6 | 515.8 | 16.9 |
| | | encephalopathy | | | | | | | | |
| | | with | | | | | | | | |
| | | biphasic | | | | | | | | |
| | | seizures | | | | | | | | |
| | | and late | | | | | | | | |
| | | reduced | | | | | | | | |
| | | diffusion, | | | | | | | | |
| | | unknown | | | | | | | | |
| | | virus | | | | | | | | |
| M | 3.0 | rotavirus | severe | recovery | 1.1 | 4.7 | 64.5 | 175.3 | 364.2 | 32.5 |
| | | encephalopathy | | | | | | | | |
| M | 3 | influenza | moderate | initial | 17.9 | 17.7 | 88.3 | 377.4 | 757.2 | 33.3 |
| | | encephalopathy | | | | | | | | |
| | | (B+) | | | | | | | | |
| M | 3 | influenza | severe | peak | 68.9 | 638.6 | 1212.0 | 323.2 | 293.5 | 52.4 |
| | | encephalopathy | | | | | | | | |
| | | (A+) | | | | | | | | |
| unknown | 3 | influenza | severe | initial | 158.5 | 1933.9 | 3226.7 | 516.1 | 811.6 | 38.9 |
| | | encephalopathy | | | | | | | | |
| F | 3.0 | acute | moderate | recovery | 13.5 | 8.9 | 31.0 | 179.2 | 880.1 | 16.9 |
| | | encephalopathy, | | | | | | | | |
| | | brain | | | | | | | | |
| | | edema, | | | | | | | | |
| | | VLCAD | | | | | | | | |
| | | deficiency, | | | | | | | | |
| | | influenza | | | | | | | | |
| F | 3.0 | rotavirus | severe | peak | 29.5 | 16.3 | 93.3 | 237.5 | 754.1 | 23.9 |
| | | encephalopathy | | | | | | | | |
| F | 3 | acute | moderate | screening | 15.4 | 8.4 | 64.1 | 285.0 | 853.1 | 25.0 |
| | | encephalopathy | | (stable) | | | | | | |
| | | with | | | | | | | | |
| | | biphasic | | | | | | | | |
| | | seizures | | | | | | | | |
| | | and late | | | | | | | | |
| | | reduced | | | | | | | | |
| | | diffusion | | | | | | | | |
| M | 3 | epileptic | moderate | screening | 29.2 | 5.5 | 63.8 | 199.9 | 764.7 | 20.7 |
| | | encephalopathy | | (stable) | | | | | | |
| F | 3 | acute | moderate | initial | 25.0 | 25.0 | 156.3 | 288.0 | 689.4 | 29.5 |
| | | encephalopathy | | | | | | | | |
| M | 3 | hypoxic | moderate | initial | 39.0 | 23.8 | 109.6 | 354.8 | 575.7 | 38.1 |
| | | ischemic | | | | | | | | |
| | | encephalopathy | | | | | | | | |
| M | 4.00 | rhabdomyolysis | mild | peak | 23.9 | 10.8 | 92.0 | 210.6 | 1032.6 | 16.9 |
| M | 4.00 | bronchitis, | moderate | screening | 18.7 | 6.2 | 28.0 | 149.4 | 775.3 | 16.2 |
| | | hypoglycemia | | (stable) | | | | | | |
| F | 4.0 | unknown | moderate | recovery | 15.9 | 14.1 | 67.7 | 188.1 | 1042.3 | 15.3 |
| | | virus | | | | | | | | |
| | | related | | | | | | | | |
| | | hepatitis | | | | | | | | |
| | | (herpangina) | | | | | | | | |
| F | 4.0 | recurrent | mild | initial | 20.4 | 8.3 | 60.0 | 409.4 | 1126.7 | 26.6 |
| | | vomiting, | | | | | | | | |
| | | hypoglycemia, | | | | | | | | |
| | | developmental | | | | | | | | |
| | | delay | | | | | | | | |
| M | 4 | liver | moderate | screening | 19.6 | 16.9 | 101.2 | 119.2 | 697.1 | 14.6 |
| | | disorder | | (stable) | | | | | | |
| F | 4.0 | hypoglycemia | mild | screening | 27.9 | 10.9 | 73.5 | 160.7 | 1054.5 | 13.2 |
| | | | | (stable) | | | | | | |
| unknown | 4 | Hypre-CK- | mild | screening | 40.2 | 7.2 | 52.2 | 138.6 | 611.1 | 18.5 |
| | | emia | | (stable) | | | | | | |
| M | 4 | GA II | unknown | screening | 33.0 | 7.3 | 89.4 | 706.4 | 1384.0 | 33.8 |
| | | suspect | | (stable) | | | | | | |
| M | 4 | hyperlactatemia | moderate | recovery | 20.2 | 7.2 | 62.9 | 274.3 | 660.0 | 29.4 |
| M | 4.0 | epilepsy, | moderate | screening | 20.1 | 0.7 | 28.1 | 120.9 | 546.9 | 18.1 |
| | | influenza | | (stable) | | | | | | |
| | | encephalopathy, | | | | | | | | |
| | | metabolic | | | | | | | | |
| | | disorder? | | | | | | | | |
| F | 4 | hypoxic | moderate | recovery | 25.1 | 27.7 | 188.5 | 278.5 | 622.4 | 30.9 |
| | | ischemic | | | | | | | | |
| | | encephalopathy | | | | | | | | |
| F | 4 | acute | moderate | initial | 40.9 | 5.1 | 64.7 | 148.8 | 582.7 | 20.3 |
| | | encephalopathy | | | | | | | | |
| F | 4 | acute | moderate | initial | 20.6 | 8.9 | 57.9 | 99.3 | 399.5 | 19.9 |
| | | encephalopathy | | | | | | | | |
| M | 5.0 | secondary | mild | recovery | 14.9 | 6.6 | 50.2 | 456.7 | 1067.8 | 30.0 |
| | | carnitine | | | | | | | | |
| | | deficiency | | | | | | | | |
| F | 5.0 | unconsciousness, | moderate | recovery | 13.1 | 3.2 | 48.0 | 111.0 | 764.2 | 12.7 |
| | | vomiting | | | | | | | | |
| F | 5.00 | hepatosplenomegaly | moderate | screening | 24.3 | 6.4 | 33.8 | 61.9 | 324.6 | 16.0 |
| | | | | (stable) | | | | | | |
| F | 5 | ketoacidosis, | moderate | initial | 2.7 | 9.6 | 55.6 | 111.7 | 676.8 | 14.2 |
| | | hypoglycemia | | | | | | | | |
| F | 5.0 | hyperlactatemia | mild | screening | 29.3 | 24.2 | 82.3 | 194.9 | 923.8 | 17.4 |
| | | | | (stable) | | | | | | |
| M | 5 | periodic | mild | screening | 19.1 | 11.3 | 85.7 | 176.8 | 1025.4 | 14.7 |
| | | paralysis | | (stable) | | | | | | |
| | | suspect, | | | | | | | | |
| | | GAII | | | | | | | | |
| | | suspect | | | | | | | | |
| F | 5 | acute | moderate | initial | 29.4 | 22.1 | 123.5 | 638.9 | 1139.9 | 35.9 |
| | | encephalopathy | | | | | | | | |
| | | with | | | | | | | | |
| | | biphasic | | | | | | | | |
| | | seizures | | | | | | | | |
| | | and late | | | | | | | | |
| | | reduced | | | | | | | | |
| | | diffusion | | | | | | | | |
| | | (B+) | | | | | | | | |
| M | 5 | acute | moderate | initial | 29.3 | 15.7 | 84.6 | 253.0 | 686.4 | 26.9 |
| | | encephalopathy | | | | | | | | |
| M | 5.0 | hypoxic | severe | unknown | 16.9 | 8.5 | 63.1 | 241.9 | 586.0 | 29.2 |
| | | ischemic | | | | | | | | |
| | | encephalopathy | | | | | | | | |
| M | 5.0 | Leigh | mild | initial | 27.5 | 16.5 | 94.3 | 184.9 | 635.4 | 22.5 |
| | | encephalopathy | | | | | | | | |
| | | (suspect) | | | | | | | | |
| M | 5 | Ifosfamide | unknown | unknown | 23.5 | 13.5 | 101.4 | 500.9 | 656.8 | 43.3 |
| | | encephalopathy | | | | | | | | |
| M | 5 | febrile | mild | peak | 19.9 | 11.3 | 64.5 | 105.4 | 659.6 | 13.8 |
| | | convulsion, | | | | | | | | |
| | | ADHD, | | | | | | | | |
| | | encephalopathy, | | | | | | | | |
| | | hypotonic | | | | | | | | |
| F | 6.0 | chronic | mild | unknown | 22.3 | 5.1 | 27.0 | 213.3 | 907.8 | 19.0 |
| | | lung | | | | | | | | |
| | | disease, | | | | | | | | |
| | | cyclic | | | | | | | | |
| | | vomiting | | | | | | | | |
| M | 6 | cyclic | mild | initial | 12.7 | 11.8 | 50.4 | 171.9 | 757.7 | 18.5 |
| | | vomiting | | | | | | | | |
| F | 6.0 | lissencephaly | moderate | screening | 24.8 | 6.9 | 71.3 | 226.6 | 639.1 | 26.2 |
| | | with | | (stable) | | | | | | |
| | | Infantile | | | | | | | | |
| | | spasms | | | | | | | | |
| F | 6 | complex | moderate | screening | 16.8 | 42.6 | 27.3 | 84.0 | 457.8 | 15.5 |
| | | partial | | (stable) | | | | | | |
| | | status | | | | | | | | |
| | | epilepsy | | | | | | | | |
| F | 6 | viral acute | moderate | recovery | 24.1 | 18.4 | 108.7 | 330.2 | 643.4 | 33.9 |
| | | encephalopathy, | | | | | | | | |
| | | hypophosphatasia | | | | | | | | |
| F | 6 | influenza | unknown | unknown | 18.5 | 5.5 | 69.9 | 140.7 | 719.1 | 16.4 |
| | | encephalopathy | | | | | | | | |
| | | suspect | | | | | | | | |
| F | 6.0 | influenza | moderate | initial | 19.8 | 9.8 | 62.7 | 394.2 | 705.4 | 35.8 |
| | | encephalopathy | | | | | | | | |
| | | (B+ | | | | | | | | |
| | | suspect) | | | | | | | | |
| M | 6.0 | influenza | moderate | recovery | 17.3 | 32.6 | 30.0 | 87.1 | 404.7 | 17.7 |
| | | encephalopathy | | | | | | | | |
| M | 6 | acute focal | moderate | screening | 33.5 | 24.2 | 139.8 | 430.7 | 1134.9 | 27.5 |
| | | bacterial | | (stable) | | | | | | |
| | | nephritis | | | | | | | | |
| | | encephalopathy | | | | | | | | |
| | | suspect | | | | | | | | |
| M | 7 | myositis | moderate | peak | 19.9 | 8.7 | 76.2 | 166.8 | 833.7 | 16.7 |
| | | due to | | | | | | | | |
| | | influenza, | | | | | | | | |
| | | rhabdomyolysis | | | | | | | | |
| M | 7.0 | VLCAD | mild | peak | 19.6 | 12.4 | 65.2 | 233.1 | 973.1 | 19.3 |
| M | 7.0 | cyclic | mild | screening | 16.2 | 6.4 | 52.0 | 116.1 | 711.8 | 14.0 |
| | | vomiting | | (stable) | | | | | | |
| M | 7 | recurrent | mild | screening | 19.6 | 9.3 | 64.3 | 390.3 | 966.6 | 28.8 |
| | | liver | | (stable) | | | | | | |
| | | dysfunction | | | | | | | | |
| F | 7 | mental | moderat | screening | 14.3 | 3.9 | 41.7 | 157.0 | 666.0 | 19.1 |
| | | deficiency, | e | (stable) | | | | | | |
| | | neurogenic | | | | | | | | |
| | | paralysis, | | | | | | | | |
| | | epilepsy | | | | | | | | |
| M | 7 | partial | moderat | screening | 14.4 | 12.0 | 48.3 | 143.0 | 649.1 | 18.1 |
| | | seizure, | e | (stable) | | | | | | |
| | | West | | | | | | | | |
| | | syndrome, | | | | | | | | |
| | | neonatal | | | | | | | | |
| | | asphyxia | | | | | | | | |
| unknown | 7 | FDPase | unknown | screening | 17.8 | 8.4 | 80.7 | 446.6 | 925.8 | 32.5 |
| | | deficiency | | (stable) | | | | | | |
| | | suspect, | | | | | | | | |
| | | ketotic | | | | | | | | |
| | | hypoglycemia | | | | | | | | |
| M | 7.0 | hyper-CK- | mild | initial | 27.9 | 37.5 | 24.5 | 113.0 | 506.8 | 18.2 |
| | | emia | | | | | | | | |
| M | 7 | acute | severe | recovery | 12.0 | 16.7 | 88.9 | 115.9 | 456.1 | 20.3 |
| | | encephalopathy | | | | | | | | |
| M | 7 | encephalopathy | moderate | screening | 2.2 | 1.5 | 31.6 | 73.1 | 527.8 | 12.2 |
| | | | | (stable) | | | | | | |
| F | 7 | acute | unknown | unknown | 11.6 | 9.0 | 55.4 | 400.7 | 769.7 | 34.2 |
| | | encephalopathy | | | | | | | | |
| M | 7 | acute | moderate | recovery | 11.1 | 4.7 | 71.5 | 139.6 | 591.5 | 19.1 |
| | | encephalopathy, | | | | | | | | |
| | | seizure | | | | | | | | |
| M | 8.0 | kidney | unknown | screening | 24.3 | 6.4 | 54.3 | 153.1 | 857.1 | 15.2 |
| | | stone | | (stable) | | | | | | |
| M | 8.00 | vomiting, | mild | initial | 15.3 | 7.9 | 19.7 | 121.4 | 669.4 | 15.4 |
| | | influenza | | | | | | | | |
| M | 8.0 | acetonemic | mild | recovery | 22.0 | 7.8 | 52.3 | 162.7 | 905.0 | 15.2 |
| | | vomiting | | | | | | | | |
| M | 8 | cyclic | mild | screening | 21.7 | 7.0 | 60.1 | 175.6 | 1008.7 | 14.8 |
| | | vomiting, a | | (stable) | | | | | | |
| | | bilious | | | | | | | | |
| | | attack | | | | | | | | |
| M | 8.0 | Asperger | mild | screening | 4.6 | 9.4 | 56.7 | 152.4 | 676.3 | 18.4 |
| | | syndrome, | | (stable) | | | | | | |
| | | epileptic | | | | | | | | |
| | | seizure, | | | | | | | | |
| | | EEG, | | | | | | | | |
| | | Roland | | | | | | | | |
| | | epilepsy | | | | | | | | |
| | | suspect | | | | | | | | |
| F | 8.00 | hyperammonemia, | moderate | peak | 11.1 | 74.3 | 292.6 | 840.7 | 25.8 | 97.0 |
| | | febrile | | | | | | | | |
| | | convulsion, | | | | | | | | |
| | | influenza A | | | | | | | | |
| M | 8.0 | influenza | moderate | screening | 16.5 | 7.1 | 68.6 | 159.5 | 752.9 | 17.5 |
| | | encephalopathy | | (stable) | | | | | | |
| F | 8 | influenza | moderate | recovery | 29.9 | 5.7 | 65.8 | 148.1 | 422.8 | 25.9 |
| | | encephalopathy | | | | | | | | |
| M | 8.0 | influenza | moderate | recovery | 29.9 | 2.9 | 97.3 | 271.9 | 607.4 | 30.9 |
| | | A+ | | | | | | | | |
| | | encephalopathy, | | | | | | | | |
| | | influenza | | | | | | | | |
| | | pneumonia | | | | | | | | |
| M | 8.0 | influenza | moderate | recovery | 22.2 | 15.0 | 40.3 | 139.2 | 627.9 | 18.1 |
| | | encephalopathy | | | | | | | | |
| F | 8 | encephalopathy, | severe | recovery | 33.1 | 5.6 | 59.5 | 160.2 | 696.5 | 18.7 |
| | | unknown | | | | | | | | |
| | | virus | | | | | | | | |
| M | 8 | acute | moderate | unknown | 49.5 | 21.3 | 129.5 | 147.1 | 283.6 | 34.1 |
| | | encephalopathy | | | | | | | | |
| M | 8 | acute | severe | peak | 29.2 | 15.1 | 126.9 | 357.2 | 859.7 | 29.4 |
| | | encephalopathy | | | | | | | | |
| M | 8 | acute | moderate | unknown | 16.3 | 16.1 | 62.8 | 162.5 | 590.0 | 21.6 |
| | | encephalopathy | | | | | | | | |
| F | 9.0 | epilepsy, | mild | screening | 16.9 | 10.3 | 75.5 | 581.0 | 1043.9 | 35.8 |
| | | asthma | | (stable) | | | | | | |
| M | 9 | acetonic | mild | initial | 14.0 | 8.5 | 66.0 | 110.6 | 670.9 | 14.2 |
| | | vomiting | | | | | | | | |
| M | 9 | fulminant | severe | peak | 31.3 | 23.7 | 130.2 | 154.9 | 491.6 | 24.0 |
| | | hepatic | | | | | | | | |
| | | failure | | | | | | | | |
| F | 9.0 | MR | mild | screening | 15.8 | 6.7 | 49.0 | 175.2 | 712.7 | 19.7 |
| | | | | (stable) | | | | | | |
| F | 9 | acute | moderate | recovery | 22.1 | 13.1 | 68.2 | 161.4 | 452.2 | 26.3 |
| | | encephalopathy | | | | | | | | |
| F | 9.5 | influenza | severe | peak | 21.0 | 7.9 | 55.6 | 214.6 | 484.7 | 30.7 |
| | | A+ | | | | | | | | |
| | | encephalopathy | | | | | | | | |
| M | 10 | cyclic | mild | screening | 22.9 | 9.0 | 76.6 | 405.6 | 998.8 | 28.9 |
| | | vomiting | | (stable) | | | | | | |
| unknown | 10.0 | recurrent | moderate | recovery | 25.0 | 7.0 | 68.5 | 415.3 | 1133.9 | 26.8 |
| | | myopathy | | | | | | | | |
| F | 10.0 | hypoxic | moderate | initial | 20.6 | 12.1 | 101.4 | 426.8 | 1005.7 | 29.8 |
| | | ischemic | | | | | | | | |
| | | encephalopathy | | | | | | | | |
| M | 10 | acute | moderate | initial | 19.2 | 19.7 | 158.5 | 243.5 | 663.6 | 26.8 |
| | | encephalopathy, | | | | | | | | |
| | | gall | | | | | | | | |
| | | stone, | | | | | | | | |
| | | choledocholithiasis | | | | | | | | |
| M | 11 | vomiting, | moderate | recovery | 16.6 | 8.2 | 67.5 | 116.0 | 840.2 | 12.1 |
| | | liver | | | | | | | | |
| | | dysfunction | | | | | | | | |
| F | 11.0 | tonic-clonic | moderate | recovery | 24.8 | 5.3 | 79.8 | 95.4 | 470.1 | 16.9 |
| | | seizure, | | | | | | | | |
| | | rash | | | | | | | | |
| F | 11 | myoclonic | mild | screening | 12.6 | 6.5 | 42.2 | 145.1 | 807.6 | 15.2 |
| | | epilepsy | | (stable) | | | | | | |
| F | 11.0 | myopathy, | moderate | recovery | 1.4 | 7.5 | 7.9 | 4.8 | 484.7 | 1.0 |
| | | rhabdomyolysis | | | | | | | | |
| F | 11 | viral acute | moderate | peak | 27.6 | 21.2 | 195.3 | 194.2 | 359.6 | 35.1 |
| | | encephalopathy | | | | | | | | |
| | | suspect | | | | | | | | |
| M | 11 | influenza | moderate | recovery | 20.8 | 10.9 | 108.7 | 731.3 | 1188.9 | 38.1 |
| | | encephalopathy | | | | | | | | |
| | | (A+) | | | | | | | | |
| M | 11 | encephalopathy, | mild | recovery | 21.6 | 10.2 | 73.8 | 335.1 | 722.6 | 31.7 |
| | | metabolic | | | | | | | | |
| | | disorder?, | | | | | | | | |
| | | influenza | | | | | | | | |
| | | B+ | | | | | | | | |
| F | 12.0 | mycoplasma | mild | peak | 2.2 | 16.9 | 83.9 | 101.5 | 504.1 | 16.8 |
| | | pneumonia | | | | | | | | |
| M | 12 | influenza | severe | initial | 18.5 | 136.9 | 826.7 | 152.4 | 217.8 | 41.2 |
| | | encephalopathy | | | | | | | | |
| | | (B+) | | | | | | | | |
| M | 12 | influenza | mild | screening | 85.0 | 13.5 | 84.4 | 235.8 | 832.1 | 22.1 |
| | | encephalopathy | | (stable) | | | | | | |
| | | (A+) | | | | | | | | |
| M | 12.0 | influenza | moderate | screening | 52.9 | 10.1 | 61.7 | 214.0 | 881.2 | 19.5 |
| | | encephalopathy | | (stable) | | | | | | |
| | | (A+) | | | | | | | | |
| F | 12 | influenza | severe | initial | 105.6 | 278.0 | 658.4 | 412.3 | 457.7 | 47.4 |
| | | encephalopathy | | | | | | | | |
| | | (B+) | | | | | | | | |
| M | 13 | carnitine | moderate | screening | 20.1 | 5.7 | 44.5 | 124.8 | 687.7 | 15.4 |
| | | deficiency, | | (stable) | | | | | | |
| | | renal | | | | | | | | |
| | | tubular | | | | | | | | |
| | | dysfunction | | | | | | | | |
| | | cardiomegaly | | | | | | | | |
| M | 13 | rhabdomyolysis | moderate | screening | 20.9 | 6.0 | 57.2 | 92.3 | 594.9 | 13.4 |
| | | | | (stable) | | | | | | |
| M | 13.0 | hematuria, | mild | screening | 19.1 | 9.7 | 51.7 | 190.7 | 806.5 | 19.1 |
| | | hyper-CK- | | (stable) | | | | | | |
| | | emia | | | | | | | | |
| F | 13 | influenza | moderate | initial | 50.8 | 49.1 | 190.5 | 278.4 | 437.6 | 38.9 |
| | | encephalopathy | | | | | | | | |
| | | (B+) | | | | | | | | |
| M | 13 | acute | mild | screening | 15.4 | 0.4 | 12.7 | 154.4 | 613.8 | 20.1 |
| | | encephalopathy | | (stable) | | | | | | |
| M | 14.0 | carnitine | mild | screening | 21.2 | 5.0 | 42.4 | 100.9 | 589.0 | 14.6 |
| | | deficiency | | (stable) | | | | | | |
| | | suspect | | | | | | | | |
| M | 14 | fatty liver | mild | screening | 18.9 | 9.2 | 68.7 | 146.7 | 655.7 | 18.3 |
| | | | | (stable) | | | | | | |
| M | 14 | metabolic | mild | screening | 18.2 | 6.4 | 41.1 | 78.4 | 525.3 | 13.0 |
| | | myopathy | | (stable) | | | | | | |
| | | suspect | | | | | | | | |
| F | 14.0 | CPA post | severe | peak | 4.2 | 23.8 | 97.3 | 101.2 | 350.5 | 22.4 |
| | | influenza A | | | | | | | | |
| M | 14.0 | fever, | severe | peak | 46.6 | 186.0 | 766.5 | 232.4 | 422.8 | 35.5 |
| | | influenza | | | | | | | | |
| | | B+ | | | | | | | | |
| | | encephalopathy, | | | | | | | | |
| | | multiple | | | | | | | | |
| | | organ | | | | | | | | |
| | | failure, | | | | | | | | |
| | | DIC | | | | | | | | |
| M | 14 | influenza | moderate | recovery | 71.4 | 9.0 | 62.4 | 90.8 | 376.8 | 19.4 |
| | | encephalopathy | | | | | | | | |
| | | (B+) | | | | | | | | |
| M | 14 | influenza | moderate | initial | 78.0 | 13.4 | 84.3 | 115.1 | 388.2 | 22.9 |
| | | encephalopathy | | | | | | | | |
| | | (B+) | | | | | | | | |
| F | 14 | acute | moderate | peak | 33.2 | 7.9 | 75.0 | 96.4 | 296.4 | 24.5 |
| | | encephalopathy | | | | | | | | |
| M | 14.2 | influenza | moderate | initial | 21.4 | 7.7 | 74.8 | 181.8 | 526.3 | 25.7 |
| | | encephalopathy | | | | | | | | |
| | | (A+) | | | | | | | | |
| M | 15.0 | muscle | mild | screening | 1.1 | 7.1 | 35.9 | 81.0 | 307.5 | 20.9 |
| | | weakness | | (stable) | | | | | | |
| LL | 15.0 | ventricular | moderate | screening | 16.9 | 3.8 | 36.3 | 51.7 | 316.4 | 14.0 |
| F | | tachycardia | | (stable) | | | | | | |
| F | 15 | influenza | moderate | initial | 20.8 | 24.5 | 84.4 | 255.6 | 329.2 | 43.7 |
| | | A+ | | | | | | | | |
| | | encephalopathy | | | | | | | | |
| | | (suspect) | | | | | | | | |
| M | 15 | acute | moderate | recovery | 15.5 | 14.7 | 77.9 | 74.5 | 296.7 | 20.1 |
| | | lymphocytic | | | | | | | | |
| | | leukemia, | | | | | | | | |
| | | encephalopathy, | | | | | | | | |
| | | hyperammonemia | | | | | | | | |
| M | 17 | hyper-CK- | moderate | peak | 19.6 | 5.6 | 46.2 | 71.4 | 396.5 | 15.3 |
| | | emia, | | | | | | | | |
| | | severe | | | | | | | | |
| | | muscle | | | | | | | | |
| | | ache | | | | | | | | |
| M | 17.0 | acute | mild | screening | 24.8 | 6.7 | 34.6 | 70.5 | 148.6 | 32.2 |
| | | encephalopathy | | (stable) | | | | | | |
| M | 20 | acute | mild | screening | 44.1 | 11.6 | 79.8 | 131.4 | 546.8 | 19.4 |
| | | encephalopathy | | (stable) | | | | | | |
| unknown | 26 | CP, | mild | screening | 15.6 | 5.6 | 48.6 | 32.6 | 204.6 | 13.7 |
| | | epilepsy, | | (stable) | | | | | | |
| | | MR, | | | | | | | | |
| | | asthma, | | | | | | | | |
| | | testicular | | | | | | | | |
| | | cancer | | | | | | | | |
| F | 26 | hyper-CK- | moderate | screening | 20.1 | 4.6 | 30.9 | 43.3 | 196.1 | 18.1 |
| | | emia | | (stable) | | | | | | |
| F | 37 | leukoencephalopathy | mild | screening | 17.2 | 4.7 | 32.5 | 105.5 | 185.5 | 36.2 |
| | | suspect | | (stable) | | | | | | |
| F | 40.0 | encephalitis, | mild | screening | 14.5 | 6.2 | 39.7 | 33.4 | 226.5 | 12.8 |
| | | hyper- | | (stable) | | | | | | |
| | | CK-emia | | | | | | | | |
| F | 41 | Ifosfamide | unknown | unknown | 43.6 | 7.5 | 45.5 | 64.9 | 204.5 | 24.1 |
| | | encephalopathy | | | | | | | | |
| M | 43 | leukoencephalopathy | mild | screening | 3.8 | 9.8 | 43.5 | 136.4 | 369.7 | 26.9 |
| | | (CADASIL | | (stable) | | | | | | |
| | | syndrome | | | | | | | | |
| | | suspect) | | | | | | | | |
| M | 54 | hyper-CK- | mild | screening | 17.9 | 5.7 | 44.5 | 86.8 | 334.3 | 20.6 |
| | | emia, | | (stable) | | | | | | |
| | | stiffness of | | | | | | | | |
| | | back | | | | | | | | |
| F | 57 | Ifosfamide | unknown | unknown | 65.0 | 11.0 | 67.3 | 93.3 | 513.0 | 15.4 |
| | | encephalopathy | | | | | | | | |
| F | 59.0 | Ifosfamide | unknown | unknown | 61.7 | 9.8 | 59.5 | 106.9 | 286.9 | 27.1 |
| | | encephalopathy | | | | | | | | |
| F | 60 | Ifosfamide | moderate | recovery | 89.7 | 12.0 | 88.0 | 62.5 | 280.7 | 18.2 |
| | | encephalopathy | | | | | | | | |
| M | 62 | leukoencephalopathy | unknown | unknown | 20.5 | 4.8 | 38.1 | 102.2 | 305.2 | 25.1 |

The data for the control patients is tabulated in Table 2.

TABLE 2
Control Patients

| | | | | | | | | Di- | Mono- | |
|---|---|---|---|---|---|---|---|---|---|---|
| | | | | Disease | ΔDiHS- | ΔDiHS- | | sulfated | sulfated | DiKS/ |
| Sex | Age | Diagnosis | Severity | stage | 0S | NS | ΔDi4S | KS | KS | Total KS |
| M | 0.1 | brain death | severe | peak | 18.6 | 27.5 | 147.0 | 106.3 | 643.3 | 14.2 |
| F | 0.0 | poor milk | mild | screening | 34.4 | 21.0 | 129.7 | 94.5 | 760.5 | 11.1 |
| | | feeding, | | (stable) | | | | | | |
| | | abnormal MRI | | | | | | | | |
| M | 0.1 | disturbance of | moderate | initial | 18.9 | 16.1 | 117.5 | 86.4 | 654.8 | 11.7 |
| | | consciousness, | | | | | | | | |
| | | hyperammonemia | | | | | | | | |
| M | 0.167 | methemoglobinemia | moderate | screening | 22.3 | 1.5 | 63.0 | 80.6 | 343.1 | 19.0 |
| | | | | (stable) | | | | | | |
| M | 0.167 | high citrulline | mild | screening | 23.6 | 17.4 | 132.9 | 166.0 | 1071.1 | 13.4 |
| | | | | (stable) | | | | | | |
| F | 0.08 | Influenza | severe | initial | 35.0 | 172.7 | 329.2 | 100.3 | 656.3 | 13.3 |
| | | negative | | | | | | | | |
| M | 0.3 | normal | severe | recovery | 25.2 | 27.7 | 152.7 | 74.4 | 1166.4 | 6.0 |
| M | 0.25 | ALTE, | moderate | recovery | 29.2 | 28.5 | 114.7 | 122.8 | 608.0 | 16.8 |
| | | hypothermia | | | | | | | | |
| F | 0.3 | apparent life- | moderate | screening | 15.4 | 9.6 | 89.4 | 100.2 | 979.0 | 9.3 |
| | | threatening | | (stable) | | | | | | |
| | | event (ALTE) | | | | | | | | |
| F | 0.3 | no finding | mild | screening | 29.6 | 14.7 | 137.2 | 157.6 | 1075.8 | 12.8 |
| | | | | (stable) | | | | | | |
| M | 0.25 | ALTE, | severe | screening | 19.4 | 19.7 | 133.4 | 161.4 | 940.7 | 14.6 |
| | | hypothermia | | (stable) | | | | | | |
| M | 0.50 | unknown | unknown | screening | 21.8 | 40.0 | 46.6 | 107.7 | 672.6 | 13.8 |
| | | | | (stable) | | | | | | |
| F | 0.1 | no finding | moderate | screening | 28.5 | 23.5 | 173.0 | 132.3 | 1171.5 | 10.1 |
| | | | | (stable) | | | | | | |
| F | 0.92 | biotin | mild | screening | 27.9 | 3.8 | 84.2 | 178.4 | 729.2 | 19.7 |
| | | deficiency | | (stable) | | | | | | |
| M | 1.0 | mitochondrial | mild | screening | 19.2 | 9.3 | 84.3 | 116.8 | 784.4 | 13.0 |
| | | diseases | | (stable) | | | | | | |
| | | suspect | | | | | | | | |
| unknown | 1 | orotic acid | unknown | screening | 26.3 | 17.9 | 172.8 | 100.6 | 992.4 | 9.2 |
| | | excretion | | (stable) | | | | | | |
| F | 1.0 | no finding | mild | screening | 18.5 | 8.3 | 63.1 | 102.0 | 934.9 | 9.8 |
| | | | | (stable) | | | | | | |
| F | 1.0 | no finding | moderate | screening | 19.9 | 11.0 | 72.3 | 124.3 | 954.4 | 11.5 |
| | | | | (stable) | | | | | | |
| F | 1.0 | short stature | mild | screening | 18.7 | 8.2 | 67.1 | 100.6 | 732.8 | 12.1 |
| | | | | (stable) | | | | | | |
| unknown | 1.0 | vitamin B1 | mild | initial | 19.1 | 9.9 | 63.2 | 169.8 | 697.4 | 19.6 |
| | | deficiency | | | | | | | | |
| | | disease | | | | | | | | |
| M | 1 | biotin | moderate | recovery | 14.1 | 15.9 | 104.1 | 174.5 | 495.0 | 26.1 |
| | | deficiency, | | | | | | | | |
| | | milk allergy | | | | | | | | |
| unknown | 1.0 | vitamin B1 | moderate | recovery | 21.6 | 7.7 | 27.7 | 62.0 | 386.1 | 13.8 |
| | | deficiency | | | | | | | | |
| | | disease | | | | | | | | |
| M | 1.00 | hyperinsulinemia | mild | initial | 19.0 | 5.4 | 32.8 | 151.6 | 845.6 | 15.2 |
| M | 2.0 | disturbance of | mild | screening | 23.1 | 7.7 | 72.4 | 133.6 | 871.2 | 13.3 |
| | | consciousness | | (stable) | | | | | | |
| M | 2.0 | CP | mild | screening | 23.4 | 10.2 | 85.0 | 156.4 | 1153.1 | 11.9 |
| | | | | (stable) | | | | | | |
| M | 3.0 | no finding | unknown | unknown | 27.0 | 10.1 | 84.0 | 137.1 | 932.5 | 12.8 |
| F | 3.0 | normal | unknown | screening | 24.1 | 7.2 | 54.5 | 363.3 | 763.3 | 32.2 |
| | | | | (stable) | | | | | | |
| M | 3.00 | metabolic | mild | screening | 23.0 | 3.3 | 69.2 | 238.7 | 687.9 | 25.8 |
| | | disorder? | | (stable) | | | | | | |
| M | 5.00 | trunkal ataxia | mild | screening | 16.6 | 4.1 | 26.1 | 230.4 | 1038.3 | 18.2 |
| | | | | (stable) | | | | | | |
| M | 7.0 | muscle ache | moderate | screening | 19.7 | 9.9 | 84.9 | 160.5 | 981.5 | 14.1 |
| | | | | (stable) | | | | | | |
| F | 8.0 | urine tandem | moderate | screening | 18.0 | 5.3 | 64.7 | 146.2 | 744.3 | 16.4 |
| | | | | (stable) | | | | | | |
| M | 9.0 | general | mild | screening | 16.1 | 5.5 | 42.0 | 162.6 | 728.7 | 18.2 |
| | | fatigue, | | (stable) | | | | | | |
| | | anorexia | | | | | | | | |
| M | 9.0 | no finding | moderate | recovery | 17.0 | 8.4 | 60.9 | 147.8 | 692.7 | 17.6 |
| M | 13 | slight fever, | mild | screening | 17.2 | 5.7 | 67.2 | 266.1 | 777.4 | 25.5 |
| | | muscle ache | | (stable) | | | | | | |
| unknown | 13.0 | no finding | mild | screening | 22.0 | 3.7 | 40.4 | 69.6 | 562.0 | 11.0 |
| | | | | (stable) | | | | | | |
| M | 13.0 | normal | unknown | unknown | 18.8 | 6.7 | 59.1 | 122.3 | 722.0 | 14.5 |
| M | 14.0 | muscle pain, | mild | screening | 20.8 | 12.0 | 56.1 | 122.7 | 640.8 | 16.1 |
| | | head pain | | (stable) | | | | | | |
| F | 14.0 | urine tandem | unknown | unknown | 16.7 | 9.1 | 38.6 | 67.8 | 392.3 | 14.7 |
| F | 15.0 | no finding | mild | screening | 9.7 | 5.5 | 35.7 | 69.0 | 374.3 | 15.6 |
| | | | | (stable) | | | | | | |
| F | 34.0 | food allergy, | unknown | unknown | 11.6 | 4.6 | 42.9 | 86.8 | 424.1 | 17.0 |
| | | hives, edema | | | | | | | | |
| F | 39.0 | OTC | mild | screening | 70.1 | 4.6 | 49.3 | 53.8 | 368.6 | 12.7 |
| | | deficiency | | (stable) | | | | | | |
| | | suspect | | | | | | | | |
| F | 52 | CP, anemia, | mild | screening | 32.4 | 6.4 | 34.3 | 77.1 | 182.8 | 29.7 |
| | | VB12 high, | | (stable) | | | | | | |
| | | hyperammonemia | | | | | | | | |
| unknown | 80.0 | normal | unknown | unknown | 4.0 | 3.9 | 30.2 | 58.3 | 174.7 | 25.0 |
| unknown | 80 | normal | unknown | unknown | 3.9 | 2.6 | 24.8 | 55.4 | 152.8 | 26.6 |

The groupings of symptoms or diagnoses are listed in Table 3. Renal and respiratory disorders are listed together because both are caused by perturbation of the endothelial glycocalyx and would thus show similar pathophysiology. “n” represents the number of patients in each condition/disorder category.

Patients with respiratory and renal disorders, epilepsy, fatty acid metabolism disorders, viral infections, liver disorders, and hypoglycemia had an elevation of some GAGs in serum; however, the sample size (n in Table 3) of most groups was too small to run statistical analyses. Only the encephalopathy groups had large enough sample sizes (n=51 for viral encephalopathy and n=69 for non-viral encephalopathy) to conduct further statistical analysis.

TABLE 3
Number of Patients with Elevated GAG Disaccharides for Each Condition

| | | Elevated | | | | Mono- | Di-sulfated | Di-sulfated |
|---|---|---|---|---|---|---|---|---|
| Condition | n= | Patients | ΔDiHS-OS | ΔDiHS-NS | ΔDi-4S | sulfated KS | KS | KS/Total KS |
| Respiratory or Renal | 22 | 8 | 3 | 1 | 2 | 0 | 3 | 4 |
| D is orders | | | | | | | | |
| Fatty Acid Metabolism | 21 | 10 | 2 | 3 | 3 | 0 | 7 | 6 |
| Disorders | | | | | | | | |
| Viral Infections | 7 | 4 | 1 | 3 | 2 | 0 | 3 | 1 |
| Vomiting Disorders | 13 | 6 | 2 | 1 | 0 | 2 | 2 | 2 |
| Liver Disorders | 18 | 11 | 6 | 3 | 3 | 1 | 3 | 4 |
| Epilepsy | 33 | 15 | 4 | 4 | 2 | 0 | 8 | 7 |
| Hypoglycemia | 22 | 13 | 4 | 1 | 2 | 3 | 8 | 6 |
| Myopathy | 12 | 1 | 1 | 0 | 0 | 1 | 1 | 0 |
| Developmental | 14 | 4 | 2 | 0 | 0 | 1 | 0 | 2 |
| Disorders | | | | | | | | |
| HyperCKemia | 12 | 2 | 2 | 1 | 0 | 0 | 0 | 0 |
| Heart Conditions | 15 | 4 | 1 | 2 | 2 | 0 | 3 | 2 |
| Acidosis | 16 | 5 | 4 | 2 | 1 | 1 | 4 | 1 |
| Viral Encephalopathy | 51 | 36 | 25 | 18 | 20 | 2 | 19 | 24 |
| Nonviral | 69 | 49 | 21 | 25 | 23 | 2 | 29 | 30 |
| Encephalopathy | | | | | | | | |

Clinical diagnoses for respiratory or renal disorders include pneumonia, asthma, bronchitis, chronic obstructive pulmonary disease (COPD), and rhabdomyolysis. Fatty acid metabolism disorders diagnoses included carnitine deficiency, Reye's syndrome, carnitine palmitoyltransferase 2 (CPT2) deficiency, medium-chain acyl-CoA dehydrogenase (MCAD) deficiency, and very-long-chain acyl-CoA dehydrogenase (VLCAD) deficiency). Viral infections included rotavirus, hand-foot-mouth disease, and influenza. Vomiting disorders were all cyclic vomiting syndrome. Clinical diagnoses for liver disorders included jaundice, hyperbilirubinemia, and liver dysfunction. Clinical diagnoses for epilepsy included epilepsy, West syndrome, tonic-clonic seizures, and febrile seizures. Heart conditions included hypertrophic cardiomyopathy, abnormal ECG (electrocardiogram also referred to as EKG), mitral regurgitation (MR), myocarditis, and ventricular tachycardia. Clinical diagnoses for acidosis included glutaric acidemia II (GAII), and methylmalonic acidemia. Viral encephalopathy viruses included respiratory syncytial virus (RSV), influenza A, influenza B, rotavirus, human herpesvirus 6 (HHV-6), and norovirus. Non-viral encephalopathy diagnoses included megalencephaly, hypoglycemia encephalopathy, epileptic encephalopathy, hypoxic-ischemic encephalopathy, Leigh syndrome, periventricular leukomalacia, ifosfamide-induced encephalopathy, acute focal bacterial nephritis (AFBN) encephalopathy, and leukoencephalopathy.

### Example 2: Analysis of Encephalopathy Groups

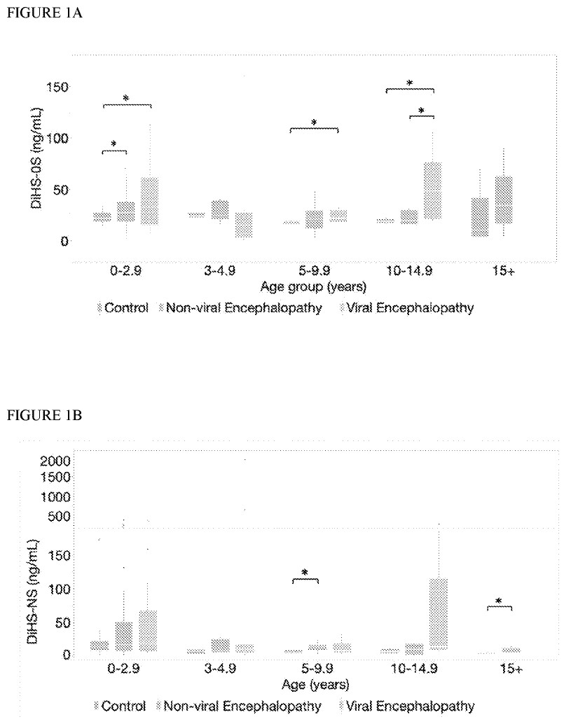

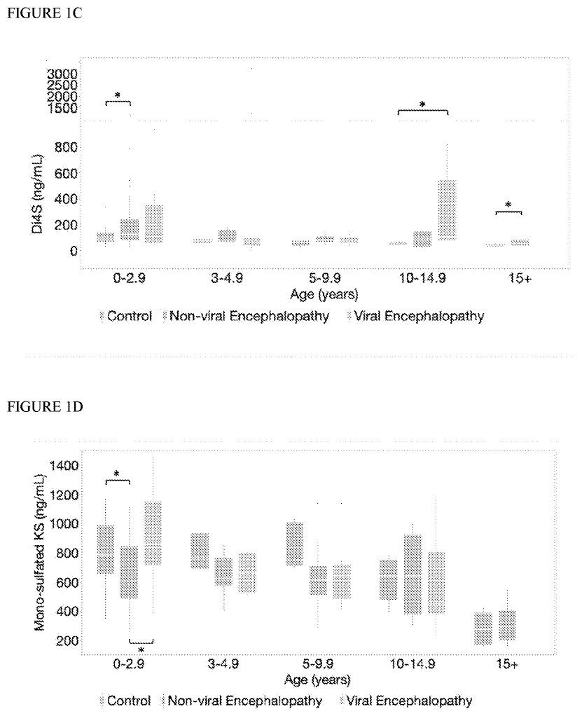

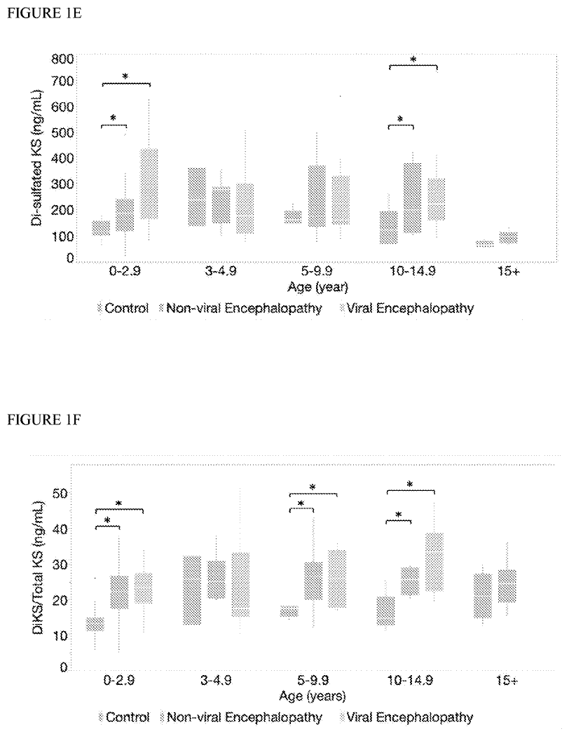

Statistical analyses on the encephalopathy groups were performed, as the patients formed the largest sample size. There is a significant difference in ΔDiHS-0S for the viral encephalopathy group compared to the control group for ages 0-2.9, 5-9.9, and 10-14.9 years old. There is also a statistical difference in the non-viral encephalopathy patients compared to the control patients for ages 0-2.9 years old (FIG. 1A). There is a significant difference in ΔDiHS-NS for the non-viral encephalopathy group compared to the control group for ages 5-9.9 and 15+ years old (FIG. 1B). A significant difference was observed in ΔDi-4S for the viral encephalopathy group compared to the control group for ages 10-14.9 years old. A significant difference was observed between the non-viral encephalopathy patients and the control patients for ages 0-2.9 and 15+ years old (FIG. 1C). Another significant difference was discerned in mono-sulfated KS between the non-viral encephalopathy group and the control group for ages 0-2.9 years old (FIG. 1D). There is a significant difference in di-sulfated KS for the viral encephalopathy group compared to the control for ages 0-2.9 and 10-14.9 years old. The data demonstrated a significant difference in di-sulfated KS for the non-viral encephalopathy group compared to the control for ages 0-2.9 and 14.9 years old (FIG. 1E). The data revealed a significant difference in the ratio of di-sulfated KS to total KS for both the viral encephalopathy group and the non-viral encephalopathy group, compared to the control group for ages 0-2.9 years, 5-9.9 years, and 10-14.9 years (FIG. 1F).

For the most part, there was no statistically significant difference between the viral and non-viral encephalopathy groups; however, there was a significant difference was revealed in ΔDiHS-0S for the 10-14.9 age group and in mono-sulfated KS for the 0-2.9 age group (FIGS. 1A and 1D).

Patients with severe forms of encephalopathy tended to have a greater level of GAG elevation. Patients with severe viral encephalopathy tended to have elevations of multiple GAGs when compared to patients with mild or moderate cases. A similar pattern was seen in patients with non-viral encephalopathy; although, two deceased patients did not show an elevation in any GAGs. While some patients with severe non-viral encephalopathy did have an elevation of multiple GAGs as with the viral encephalopathy group, the evidence was less conclusive for this group.

Furthermore, patients having severe forms of renal and respiratory conditions, viral infections, liver disorders, and acidosis seemed to have a higher prevalence of GAG elevation, and multiple GAGs were elevated in patients having these conditions. No evidence was seen that the severity of the condition or disorder effects GAG expression for fatty acid metabolism disorders, epilepsy, hypoglycemia, or heart disorders. There were no patients with severe cases for vomiting disorders, myopathy, developmental disorders, and hyperCKemia.

Out of 198 DBS samples, two samples provided a significant elevation of specific GAGs. The sample with MPS II showed that the concentration levels of Di-0S, HS-0S, HS-NS, mono-sulfated KS, and di-sulfated KS were 30.9 ng/mL, 141.6 ng/mL, 26.04 ng/mL, 146.0 ng/mL, and 35.6 ng/mL, respectively (Stapleton et al., 2020). The HS-0S and HS-NS levels were above the established cutoff values of 90 ng/mL and 23 ng/mL, respectively (Kubaski et al., 2016). Another DBS sample was derived from the extremely premature infant with an extremely low birth weight infant (birth weight; 582 g at 24 gestational weeks, female). The concentration levels of Di-0S, HS-0S, HS-NS, mono-sulfated KS, and di-sulfated KS were 137.3 ng/ml, 104.6 ng/ml, 12.6 ng/ml, 187.6 ng/ml, and 39.2 ng/ml, respectively. The Di-0S and HS-0S levels were above the established cutoff values of 88 ng/mL and 90 ng/mL, respectively.

### Discussion for Examples 1 and 2

The study demonstrated unexpectedly the elevation of blood GAGs in encephalopathy and other metabolic disorders in childhood and in the extremely premature infant and not just in patients having MPS.

The encephalopathy groups had larger sample sizes, and, therefore, statistical analyses were run on viral encephalopathy, non-viral encephalopathy, and a control group. Serum GAG levels are related to encephalopathy (FIGS. 1A-1F). However, there was a lack of conclusive evidence across age groups since some age groups included relatively small numbers of patient and/or control samples. More sample analysis would be required across age groups. Encephalopathy is any brain disease or injury that affects the structure or function of the brain. Many events can cause encephalopathy, including infection, tumor, or stroke. While there are no previous studies concerning serum GAG levels in encephalopathy as a whole, there are some studies on these underlying causes.

The data herein include that the extremely premature infant provided a significant elevation of DS and HS in the DBS sample. DS level was much higher than that in a severe form of MPS II. The indexed case had normal development after birth at 3 years of age without any brain damage. It is of great interest to understand whether premature brain and body at the developing stage need more specific GAGs with a high level of GAGs in blood or not.

There are several limitations for the current study, including that it was a retrospective study, and the sample number in most groups had small numbers, affecting the statistical analysis. Additionally, encephalopathy is often caused by an underlying disease or condition, so it is unclear whether it is the underlying cause affecting GAG levels or encephalopathy itself. While it does have limitations, this study provides insight into the level of GAGs in blood associated with various diseases, specifically in pediatric patients following the brain-associated disease. The data demonstrate that disease and GAG levels do influence each other.

### Conclusion for Examples 1 and 2

A variety of diseases and conditions elevate GAGs in serum. Both viral and non-viral encephalopathies are associated with elevated GAG levels. There is also a possible relationship between the elevation of specific GAGs and other diseases, making GAGs useful biomarkers for a variety of conditions beyond MPS.

### Example 3: Analysis Glycosaminoglycans (GAGs) in Cerebrospinal Fluids (CSF)

Cerebrospinal fluid (CSF) samples were obtained with informed consent from 12 subjects. Eight (8) of the subjects were controls and four (4) of the subjects were diagnosed with bacterial infections. The demographics of the control subjects and the subjects with a bacterial infection are provided in Tables 4 and 5, respectively.

TABLE 4
Characteristics of 8 control subjects

| | Age | ΔDiHS- | ΔDiHS- | | Monosulfated | Disulfated | DiKS/total |
|---|---|---|---|---|---|---|---|
| Diagnosis | (year) | 0S | NS | ΔDi4S | KS | KS | KS |
| Control | 0.6 | 21.36 | 2.98 | ND | 41.60 | 14.72 | 26.14 |
| Control | 9 | 23.78 | 3.34 | ND | 62.54 | 11.17 | 15.15 |
| Control | 0.25 | 21.05 | 3.11 | 4.58 | 59.96 | 15.36 | 20.39 |
| Control | 6 | 23.75 | 2.62 | 3.50 | 55.17 | 20.29 | 26.89 |
| Control | 6 | 24.48 | 3.02 | ND | 74.31 | 21.40 | 22.36 |
| Control | 3 | 23.61 | 2.87 | 0.35 | 39.30 | 4.45 | 10.17 |
| Control | 3 | 27.09 | 2.80 | 0.14 | 43.81 | 14.50 | 24.87 |
| Control | 9 | 21.42 | 1.28 | 0.05 | 28.46 | 11.52 | 28.81 |
| Avg | | 23.32 | 2.75 | 1.72 | 50.64 | 14.18 | 21.85 |
| SD | | 2.02 | 0.63 | 2.15 | 14.92 | 5.37 | 6.38 |

TABLE 5
Characteristics of 4 subjects with bacterial infections

| | Age | ΔDiHS- | ΔDiHS- | | Monosulfated | Disulfated | DiKS/total |
|---|---|---|---|---|---|---|---|
| Diagnosis | (year) | 0S | NS | ΔDi4S | KS | KS | KS |
| Infection | 0 | 37.32 | 2.31 | 0.00 | 91.49 | 18.50 | 16.82 |
| Sepsis | 0.08 | 57.40 | 3.98 | 3.03 | 152.29 | 18.85 | 11.01 |
| Bacterial | 0.75 | 59.15 | 5.06 | 7.96 | 142.91 | 20.17 | 12.37 |
| Meningitis | | | | | | | |
| Bacterial | 0.75 | 53.77 | 2.67 | 1.50 | 55.08 | 11.97 | 17.85 |
| Meningitis | | | | | | | |
| Avg | | 51.91 | 3.51 | 3.12 | 110.44 | 17.37 | 14.51 |
| SD | | 9.98 | 1.26 | 3.45 | 45.57 | 3.67 | 3.33 |

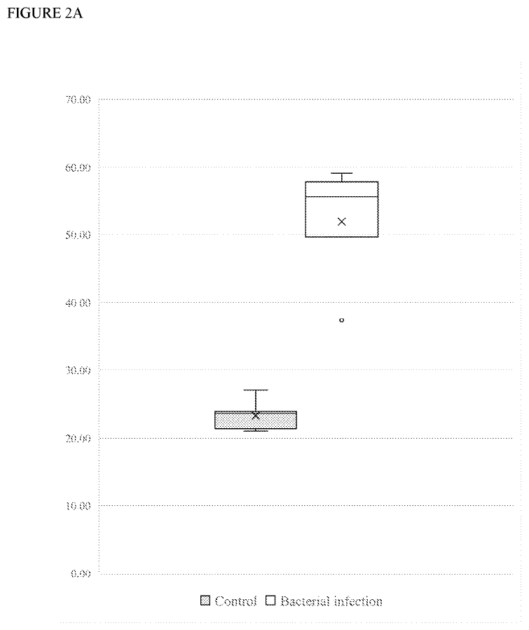

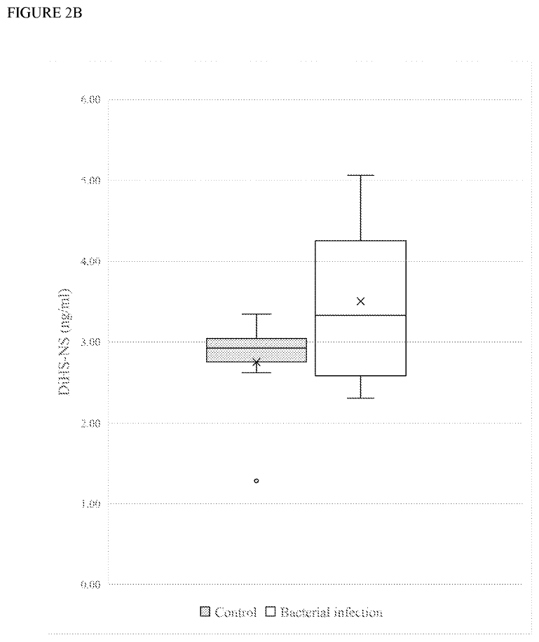

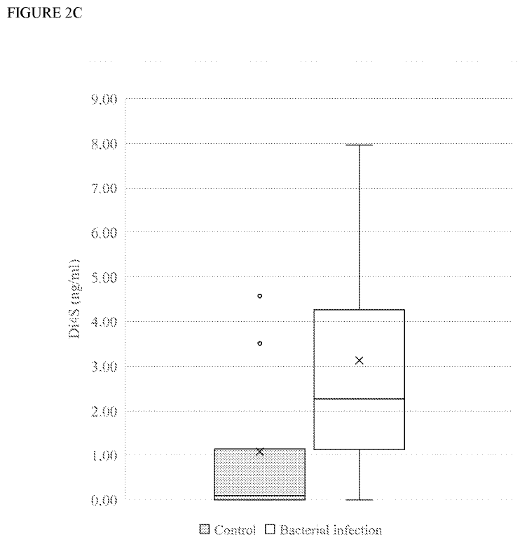

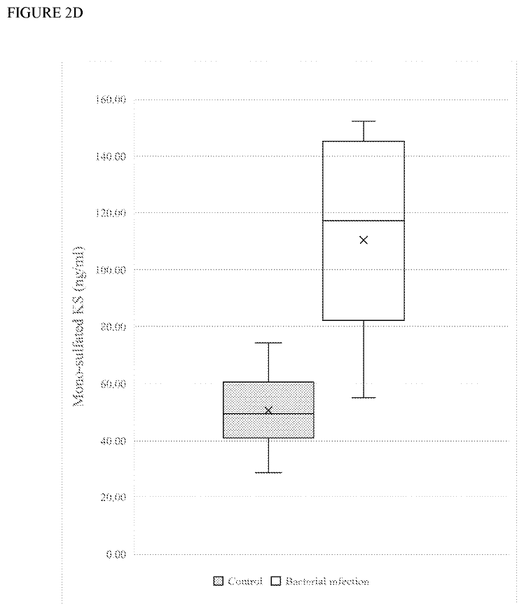

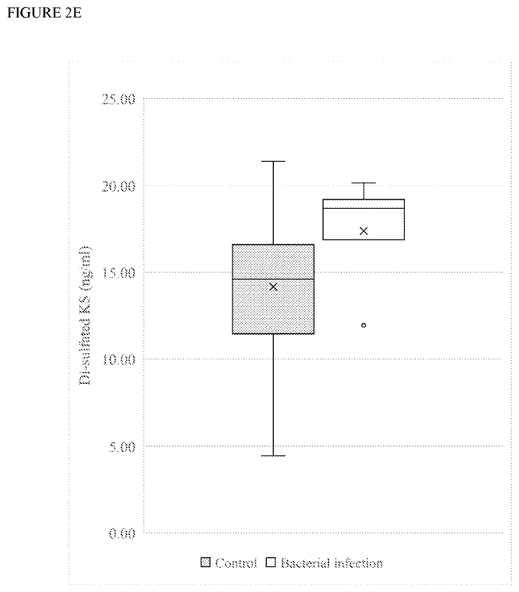

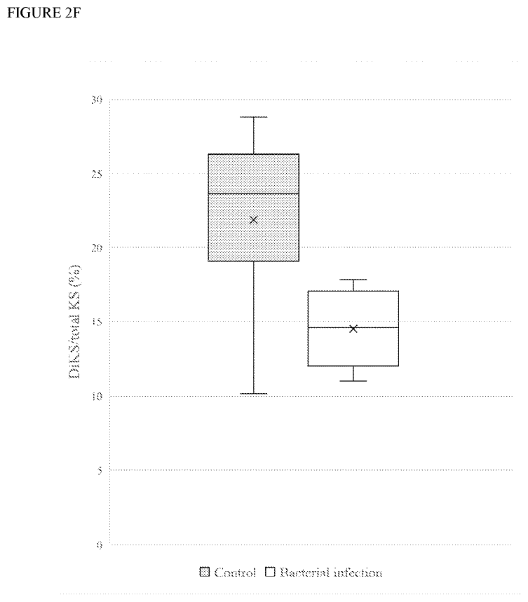

Glycoaminoglycans (GAGs) were analyzed in the CSF samples as described in Examples 1 and 2. The data are shown in Table 6, and in FIGS. 2A to 2F.

TABLE 6

| | | | | | | Di- | |
|---|---|---|---|---|---|---|---|
| | | ΔDiHS- | ΔDiHS- | | Mono- | sulfated | |
| | | 0S | NS | ΔDi4S | sulfated KS | KS | DiKS/total |
| Sample | | (ng/ml) | (ng/ml) | (ng/ml) | (ng/ml) | (ng/ml) | KS (%) |
| Controls | Avg | 23.32 | 2.75 | 1.72 | 50.64 | 14.18 | 21.85 |
| Bacterial | Avg | 51.91 | 3.51 | 3.12 | 110.44 | 17.37 | 14.51 |
| infections | | | | | | | |
| (sepsis, | | | | | | | |
| meningitis) | | | | | | | |
| Controls | SD | 2.02 | 0.63 | 2.15 | 14.92 | 5.37 | 6.38 |
| Bacterial | SD | 9.98 | 1.26 | 3.45 | 45.57 | 3.67 | 3.33 |
| infections | | | | | | | |
| (sepsis, | | | | | | | |
| meningitis) | | | | | | | |

All GAG analysis showed that the bacterial infection group had an elevation of each specific GAG (DiHS-0S, DiHS-NS, Di4S, mono-sulfated KS, di-sulfated KS). DiHS-0S in the bacterial infection group was significantly higher than that in the control group. See FIG. 2A. This finding demonstrates that DiHS-0S in CSF can be a biomarker for bacterial infection. These data are the first report that GAGs in CSF in infectious diseases can be a biomarker.

### REFERENCES

1. Miyata, S., Kitagawa, H. Formation and remodeling of the brain extracellular matrix in neural plasticity: Roles of chondroitin sulfate and hyaluronan. Biochim. Biophys. Acta. Gen. Subj. 2017, 1861 (10), 2420-2434.
2. Caterson, B., Melrose, J. Keratan sulfate, a complex glycosaminoglycan with unique functional capability. Glycobiology 2018, 28 (4), 182-206.
3. Zimmermann, D. R., Dours-Zimmermann, M. T. Extracellular matrix of the central nervous system: from neglect to challenge. Histochem. Cell. Bio. 2008, 130, 635-653.
4. Smith, P. D., Coulson-Thomas, V. J., Foscarin, S., Kwok, J. C., Fawcett, J. W., GAG-ing with the neuron: The role of glycosaminoglycan patterning in the central nervous system. Exp. Neurol. 2015, 274 (Pt B), 100-114.
5. Thomsen, M. S., Routhe, L. J., Moos, T. The vascular basement membrane in the healthy and pathological brain. J. Cerebr. Blood F. Met. 2017, 37 (10): 3300-3317.
6. Kubaski, F., Mason, R. W., Nakatomi, A., Shintaku, H., Xie, L., van Vies, N. N., Church, H., Giugliani, R., Kobayashi, H., Yamaguchi, S., Suzuki, Y., Orii, T., Fukao, T., Montaño, A. M., Tomatsu, S. Newborn screening for mucopolysaccharidoses: a pilot study of measurement of glycosaminoglycans by tandem mass spectrometry. J. Inherit. Metab. Dis. 2017, 40(1): 151-158.
7. Stapleton, M., Kubaski, F., Mason, R. W., Shintaku, H., Kobayashi, H., Yamaguchi, S., Taketani, T., Suzuki, Y., Orii, K., Orii, T., Fukao, T., Tomatsu, S. Newborn screening for mucopolysaccharidoses: Measurement of glycosaminoglycans by LC-MS/MS. Mol. Genet. Metab. Rep. 2020, 22, 100563.
8. Pihtili, A., Bingol, Z., Kiyan, E. Serum endocan levels in patients with stable COPD. Int. J. Chron. Obstruct. Pulmon. Dis. 2018, 13, 3367-3372.
9. Nikaido, T., Tanino, Y., Wang, X., Sato, S., Misa, K., Fukuhara, N., Sato, Y., Fukuhara, A., Uematsu, M., Suzuki, Y., Kojima, T., Tanino, M., Endo, Y., Tsuchiya, K., Kawamura, I., Frevert, C. W., Munakata, M. Serum Syndecan-4 as a possible biomarker in patients with acute pneumonia. J. Infect. Dis. 2015, 212 (9), 1500-1508.
10. Schmidt, E. P., Li, G., Li, L., Fu, L., Yang, Y., Overdier, K. H., Douglas, I. S., Lindhardt, R. J. The circulating glycosaminoglycan signature of respiratory failure in critically ill adults. J. Biol. Chem. 2014, 289 (12), 8194-8202.
11. Nelson, A., Berkestedt, I., Bodelsson, M. Circulating glycosaminoglycans species in septic shock. Acta. Anaesthesiol. Scand. 2014, 58, 36-43.
12. Grundmann, S., Fink, K., Rabadzhieva, L., Bourgeois, N., Schwab, T., Moser, M., Bode, C., Busch, H. J. Perturbation of the endothelial glycocalyx in post cardiac arrest syndrome. Resuscitation 2012, 83, 715-720.
13. Khan, S. A., Mason, R. W., Giugliani, R., Orii, K., Fukao, T., Suzuki, Y., Yamaguchi, S., Kobayashi, H., Orii, T., Tomatsu, S. Glycosaminoglycans analysis in blood and urine of patients with mucopolysaccharidosis. Mol. Genet. Metab. 2018, 125 (1-2), 44-52.
14. Tomatsu, S., Shimada, T., Mason, R. W., Kelly, J., LaMarr, W. A., Yasuda, E., Shibata, Y., Futatsumori, H., Montano, A. M., Yamaguchi, S., Suzuki, Y., Orii, T. Assay for glycosaminoglycans by Tandem Mass Spectrometry and its applications. J. Anal. Bioanal. Tech. 2014, 2014 (Suppl 2), 006.
15. Kubaski, F., Mason, R. W., Nakatomi, A., Shintaku, H., Xie, L., van Vlies, N. N., Church, H., Giugliani, R, Kobayashi, H., Yamaguchi, S., Suzuki, Y., Orii, T., Fukao, T., Montano, A. M., Tomatsu, S. Newborn screening for mucopolysaccharidoses: a pilot study of measurement of glycosaminoglycans by tandem mass spectrometry. J. Inherit. Metab. Dis. 2017, 40, 151-158.
16. Kubaski, F., Suzuki Y., Orii, K., Giugliani, R., Church, H. J., Mason, R. W., Dung, V. C., Ngoc, C. T., Yamaguchi, S., Kobayashi, H., Girisha, K. M., Fukao, T., Orii, T., Tomatsu, S., Glycosaminoglycan levels in dried blood spots of patients with mucopolysaccharidoses and mucolipidoses. Mol. Genet. Metab. 2017, 120, 247-254.
17. Shimada, T., Tomatsu, S., Mason, R. W., Yasuda, E., Mackenzie, W. G., Hossain, J., Shibata, Y., Montano, A. M., Kubaski, F., Giugliani, R., Yamaguchi, S., Suzuki, Y., Orii, K. E., Fukao, T., Orii, T. Di-sulfated keratan sulfate as a novel biomarker for mucopolysaccharidosis II, IVA, and IVB. JIMD Rep. 2015, 21, 1-13.
18. Shimada, T., Tomatsu, S., Yasuda, E., Mason, R. W., Mackenzie, W. G., Shibata, Y., Kubaski, F., Giugliani, R., Yamaguchi, S., Suzuki, Y., Orii, K., Orii, T. Chondroitin 6-sulfate as a novel biomarker for mucopolysaccharidosis IVA and VII. JIMD Rep. 2014, 16, 15-24.
19. Leistner, C. M., Gruen-Bernhard, S., Glebe, D. Role of glycosaminoglycans for binding and infection of hepatitis B virus. Cell Microbiol. 2008, 10 (1), 122-133. doi:10.1111/j.1462-5822.2007.01023.x
20. WuDunn, D., Spear, P. G. Initial interaction of herpes simplex virus with cells is binding to heparan sulfate. J Virol. 1989, 63 (1), 52-58.
21. Scarpa, M., Lourengo, C. M., Amartino, H. Epilepsy in mucopolysaccharidosis disorders. Mol. Genet. Metab. 2017, 122, 55-61.
22. Okamoto, M., Sakiyama, J., Mori, S., Kurazono, S., Usui, S., Hasegawa, M., Oohira, A. Kainic acid-induced convulsions cause prolonged changes in the chondroitin sulfate proteoglycans neurocan and phosphacan in the limbic structures. Exp. Neurobiol. 2003, 184, 179-195.
23. DellaValle B, Hasseldam H, Johansen F F, Iversen H K, Rungby J, Hempel C. Multiple soluble components of the glycocalyx are increased in patient plasma After ischemic stroke. Stroke 2019, 50 (10), 2948-2951.
24. Li, J., Li, J. P., Zhang, X., Lu, Z., Yu, S. P., Wei, L. Expression of heparanase in vasculature cells and astrocytes of the mouse brain after focal cerebral ischemia. Brain Research 2012, 1433, 137-144.
25. McKeon, R. J., Schreiber, R. C., Rudge, J. S., Silver, J. Reduction of neurite outgrowth in a model of glial scarring following CNS injury is correlated with the expression of inhibitory molecules on reactive astrocytes. J. Neurosci. 1991, 11, 3398-3411.
26. Kato, Y., Hayatsu, N., Kaneko, M. K., Ogasawara, S., Hamano, T., Takahashi, S., Nishikawa, R., Matsutani, M., Mishima, K., Narimatsu, H. Increased expression of highly sulfated keratan sulfate synthesized in malignant astrocytic tumors. Biochem. Biophys. Res. Commun. 2006, 369, 1041-1046.
27. Leadbeater, W. E., Gonzalez, A-M., Logaras, N., Berry, M., Turnbull, J. E., Logan, A. Intracellular trafficking in neurons and glia of fibroblast growth factor-2, fibroblast growth factor receptor 1 and heparan sulphate proteoglycans in the injured adult rat cerebral cortex. J. Neurochem. 2006, 96, 1189-1200.

While the disclosure has been disclosed with reference to specific embodiments, it is apparent that variations of this disclosure may be devised by others skilled in the art without departing from the true spirit and scope used in the practice of the disclosure. The appended claims are intended to be construed to include all such embodiments and equivalent variations.

1. A method of treating a subject having a condition identified as having an elevated glycosaminoglycan (GAG) level comprising:
a) administering at least one therapeutic treatment to the subject;
b) monitoring the subject administered the therapeutic treatment for a reduction in a GAG level at least once to determine if the at least one therapeutic treatment reduces the GAG level;
c) administering an additional therapeutic treatment to the subject; wherein the elevated GAG level is determined by enzymatic digestion of the GAG present in a biological sample to obtain disaccharides and determining the level of the disaccharides relative to normal.

2. The method of claim 1, wherein the disaccharide is selected from the group consisting of: 2-deoxy-2-sulfamino-4-(4-deoxy-a-L-threo-hex-4-enopyranosyluronic acid)-D-glucose (ΔDiHS-NS), 2-acetamido-2-deoxy-4-O-(4-deoxy-a-L-threo-hex-4-enopyranosyluronic acid)-D-glucose (ΔDiHS-0S), 2-acetamido-2-deoxy-4-O-(4-deoxy-a-L-threo-hex-4-enopyranosyluronic acid)-4-O-sulfo-D-glucose (ΔDi-4S; DS), mono-sulfated KS (Galβ1-4GlcNAc(6S)), and di-sulfated KS (Gal(6S)β1-4GlcNAc(6S)).

3. The method of claim 1, wherein the condition is selected from a respiratory condition, a renal disorder, a fatty acid metabolism disorder, a viral infection, a vomiting disorder, a liver disorder, epilepsy, hypoglycemia, myopathy, a developmental disorder, a hyperCKemia, a heart condition, acidosis, a viral encephalopathy, and a non-viral encephalopathy.

4. The method of claim 1, wherein the biological sample is a blood sample or cerebrospinal fluid (CSF).

5. A method of diagnosing severity of a condition severity in a subject in need thereof comprising:
a) measuring at least one GAG level that is elevated outside the normal range in a biological sample obtained from the subject; and
b) diagnosing condition severity as a patient having at least one GAG level two standard deviations above the mean of a control patient.

6. The method according to claim 5, wherein the biological sample is a body fluid selected from blood, plasma, serum, urine, and/or CSF.

7. The method, according to claim 5, further comprising administering at least one treatment for the condition severity diagnosed.

8. A method of treating a subject with a disease or disorder having an elevated glycosaminoglycan (GAG) level comprising:
a) assessing the level of at least one disaccharide obtained from a glycosaminoglycan in a biological sample containing glycosaminoglycans obtained from the subject that does not have a mucopolysaccharidosis (MPS);
b) determining the subject may be afflicted with a non-MPS disease or disorder when the level of the at least one disaccharide is elevated compared to the level of the same disaccharide in a biological sample from a control;
and
c) administering at least one treatment for the non-MPS disease or disorder to the subject.

9. The method according to claim 8, wherein the disease or disorder is at least one disease or disorder selected from the group consisting of: a respiratory condition, a renal disorder, a fatty acid metabolism disorder, a viral infection, a vomiting disorder, a liver disorder, epilepsy, hypoglycemia, myopathy, a developmental disorder, a hyperCKemia, a heart condition, acidosis, and encephalopathy.

10. The method according to claim 8, wherein the disease or disorder is at least one disease or disorder selected from the group consisting of: pneumonia, asthma, bronchitis, chronic obstructive pulmonary disease (COPD), rhabdomyolysis. carnitine deficiency, Reye's syndrome, carnitine palmitoyltransferase 2 (CPT2) deficiency, medium-chain acyl-CoA dehydrogenase (MCAD) deficiency, very-long-chain acyl-CoA dehydrogenase (VLCAD) deficiency, rotavirus infection, hand-foot-mouth disease viral infection, influenza infection, cyclic vomiting syndrome, jaundice, hyperbilirubinemia, liver dysfunction, epilepsy, West syndrome, tonic-clonic seizures, febrile seizures, hypertrophic cardiomyopathy, abnormal ECG, mitral regurgitation (MR), myocarditis, ventricular tachycardia, glutaric acidemia II (GAII), methylmalonic acidemia. respiratory syncytial virus (RSV), influenza A, influenza B, rotavirus, human herpes virus 6 (HHV-6), Norovirus, .megalencephaly, hypoglycemia encephalopathy, epileptic encephalopathy, hypoxic ischemic encephalopathy, Leigh syndrome, periventricular leukomalacia, ifosfamide-induced encephalopathy, acute focal bacterial nephritis (AFBN) encephalopathy, and leukoencephalopathy.

11. The method according to claim 8, wherein the disease or disorder is encephalopathy.

12. The method according to claim 9, wherein the disease or disorder is viral encephalopathy.

13. The method according to claim 12, wherein the at least one elevated disaccharide is selected from ΔDiHS-0S, ΔDi-4S, di-sulfated KS and the ratio of di-sulfated KS to total KS.

14. The method according to claim 12, wherein the disease or disorder is non-viral encephalopathy.

15. The method according to claim 14, wherein the at least one elevated disaccharide is selected from ΔDiHS-0S, ΔDiHS-NS, ΔDi-4S, mono-sulfated KS, di-sulfated KS, and the ratio of di-sulfated KS to total KS.

16. The method, according to claim 9, wherein the control comprises an age-matched control.

17. The method, according to claim 8, wherein the disease or disorder is a bacterial infection.

18. The method, according to claim 17, wherein the bacterial infection is sepsis or meningitis.

19. The method, according to claim 17, wherein the at least one elevated disaccharide is ΔDiHS-0S.

20. The method, according to claim 9, wherein the biological sample is a body fluid selected from blood, plasma, serum, urine and/or cerebrospinal fluid (CSF).
